(19) United States
(12) Patent Application Publication
(10) Pub. No.: US 2005/0161184 A1
(43) Pub. Date: Jul. 28, 2005

(54) Amphoteric and cationic fluorescent whitening agents

(76) Inventors: Goetz Scheffler, Grenzach-Wyhlen (DE); Peter Rohringer, Schonenbuch (CH); Rene Schlatter, Basel (CH); Ted Deisenroth, Brookfield, CT (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/508,444

(22) PCT Filed: Mar. 13, 2003
(86) PCT No.: PCT/EP03/02620

(30) Foreign Application Priority Data
Mar. 19, 2002 (EP) 02405211.0

(51) Int. Cl. C07D 413/14; C07D 43/14

(52) U.S. Cl. 162/162; 544/193.2; 544/112

(57) ABSTRACT

A compound of the formula wherein A₁ and A₂ each, independently of one another, represent —SO₃ or —SO₃M, where M represents hydrogen, an alkaline or alkaline earth metal, ammonium or alkylammonium, B₁ and B₂ each, independently of one another, represent the moiety in which R₁ represents hydrogen, a straight-chain C₁-C₁₂alkyl or branched C₃-C₁₂alkyl group which C₂-C₁₂alkyl and C₃-C₁₂alkyl group, respectively, may be interrupted by one or two heteroatoms and is unsubstituted or substituted by one or two —OH, —OC₁-C₄alkyl, —NH₂, —NHC₁-C₄alkyl, —N(C₁-C₄alkyl)₂, -N-pyrrolidino, -N-piperidino, -N-morpholino or —N⁺(C₁C₄alkyl)₃ groups and R₂ represents C₁-C4alkyl, C₂-C₄hydroxyalkyl, —CH₂CONH₂, —CH₂OOOH or —CH₂OOO C₁C₄alkyl or, alternatively, B₁ and B₂ each, independently of one another, represent a group of the formula in which R₃, R₄ and R₅ each, independently of each other, represent hydrogen, C₁-C₄alkyl, C₂-C₄hydroxyalkyl, the group —X′—NR₆R₇ or the group —X′—N⁺R₃R₆R₇, whereby at least one of the substituents R₄ and/or R₅ represents —X′—NR₆R₇ or —X′—N⁺R₃R₆R₇, X and X′ each, independently of each other, represent a straight-chain C₂-C₈alkylene or branched C₃-Cealkylene chain, which is unsubstituted or substituted by one or two —OH or —C(═O)— groups, R₆ and R₇ each, independently of each other, represent hydrogen, C₁-C₄alkyl or, together with the nitrogen atom to which they are bound, complete a pyrrolidino, piperidino or morpholino ring and R₂ is as previously defined and each D₁ and D₂, independently of one another, are either defined as for B₁ and B₂ or represent halogen, —NH₂, C₁-C₄monoalkyl- or dialkylamino, said alkyl groups being unsubstituted or substituted by C₁-C₄alkoxy, amino, mono- or di-C₁-C₄alkylamino or tri-C₁-C₄alkylammonium; C₂-C₄hydroxyalkylamino, C₂ C₄di(hydroxyalkyl)amino, anilino, an aniline monosulphonic acid or sulphonamide residue or a 5- or 6-membered, saturated heterocyclic ring or, alternatively, mixtures of compounds of formula (1), a process for their preparation and the use thereof as fluorescent whitening agents, especially for paper.

The present invention relates to novel amphoteric and cationic bis-triazinylaminostilbene fluorescent whitening agents (FWA's), a process for their preparation and the use thereof for fluorescent whitening of synthetic or natural organic materials, in particular, paper.

The most commonly used types of fluorescent whitening agent for the fluorescent whitening of paper are those belonging to the class of di-, tetra- or hexasulphonic acid derivatives of bis-triazinylaminostilbenes, which are anionic in nature. Modern paper-making techniques, however, generally employ cationic polymers as assistants, for example, as retention agents or dewatering aids, in particular, during the preparation of recycling papers, which, most probably contain residual amounts of anionic FWA's. The presence of cationic polymers, however, results in quenching of the fluorescence of anionic FWA's, which is clearly disadvantageous. Consequently, there is a need for a type of FWA, which is not quenched by such polymers and, in addition is combinable with anionic FWA's.

Surprisingly, it has now been found that certain novel amphoteric and also cationic FWA's are neither detrimentally affected by the presence of cationic polymers nor by the presence of residual amounts of anionic FWA's and also exhibit excellent whitening properties when applied to paper.

Accordingly, the present invention provides novel compounds of the formula 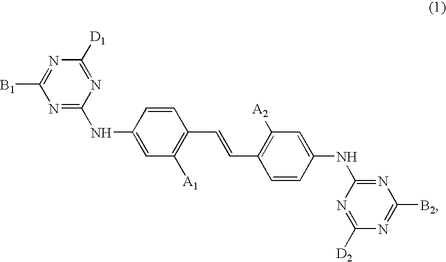
wherein
A₁ and A₂ each, independently of one another, represent —SO₃⁻ or —SO₃M, where
M represents hydrogen, an alkaline or alkaline earth metal, ammonium or alkyl ammonium,
B₁ and B₂ each, independently of one another, represent the moiety 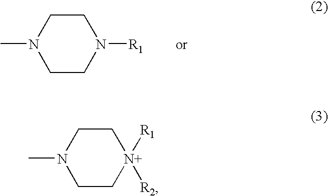
   in which
R₁ represents hydrogen, a straight-chain C₁-C₁₂alkyl or branched C₃-C₁₂alkyl group which C₂-C₁₂alkyl and C₃-C₁₂alkyl group, respectively, may be interrupted by one or two heteroatoms and is unsubstituted or substituted by one or two —OH, —OC₁-C₄alkyl, —NH₂, —NHC₁-C₄alkyl, —N(C₁-C₄alkyl)₂, -N-pyrrolidino, -N-piperidino, -N-morpholino or —N⁺(C₁-C₄alkyl)₃ groups and
R₂ represents C₁-C₄alkyl, C₂-C₄hydroxyalkyl, —CH₂CONH₂, —CH₂COOH or —CH₂COO C₁-C₄alkyl or, alternatively,
B₁ and B₂ each, independently of one another, represent a group of the formula 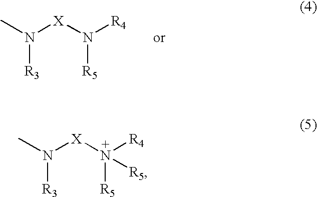
   in which
R₃, R₄ and R₅ each, independently of each other, represent hydrogen, C₁-C₄alkyl, C₂-C₄hydroxyalkyl, the group —X′—NR₆R₇ or the group —X′—N⁺R₃R₆R₇, whereby at least one of the substituents R₄ and/or R₅ represents —X′—NR₆R₇ or —X′—N⁺R₃R₆R₇,
X and X′ each, independently of each other, represent a straight-chain C₂-C₈alkylene or branched C₃-C₈alkylene chain, which is unsubstituted or substituted by one or two —OH or —C(═O)— groups,
R₆ and R₇ each, independently of each other, represent hydrogen, C₁-C₄alkyl or, together with the nitrogen atom to which they are bound, complete a pyrrolidino, piperidino or morpholino ring and
R₂ is as previously defined and each
D₁ and D₂, independently of one another, are either defined as for B₁ and B₂ or represent halogen, —NH₂, C₁-C₄monoalkyl- or dialkylamino, said alkyl groups being unsubstituted or substituted by C₁-C₄alkoxy, amino, mono- or di-C₁-C₄alkylamino or tri-C₁-C₄alkylammonium;
C₂-C₄hydroxyalkylamino, C₂-C₄di(hydroxyalkyl)amino, anilino, an aniline monosulphonic acid or sulphonamide residue or a 5- or 6-membered, saturated heterocyclic ring or, alternatively, mixtures of compounds of formula (1).

Amphoteric compounds of formula (1) may exist either in the form of an internal or external salt. Thus, for example, in the case in which M in A₁ and/or A₂ represents hydrogen, compound (1) may exist as an equilibrium mixture of a neutral molecule and of a zwitterion, wherein A₁ and/or A₂ represent —SO₃⁻, whilst the proton resides on the amine residues of substiuents B₁ and/or B₂, i.e. at least one of R₁ and R₂ in formula (3) and at least one of R₂, R₄ and R₅ in formula (5) represents hydrogen. Alternatively, such a compound may also be present in the form of an external salt, for example, where, in formula (1) A₁ and/or A₂ represents —SO₃M and a proton resides on the amine residues of substiuents B₁ and/or B₂ as described above, a further anion An⁻ must also be present. In this case and also in the case of cationic derivatives carrying excess positive charge, the anion An⁻ is a colourless anion derived from an inorganic or from an organic acid.

Typical examples of such anionic radicals include halide, e.g. chloride, bromide or iodide, sulphate, methyl sulphate, boron tetrafluoride, aminosulphonate, perchlorate, carbonate, bicarbonate, phosphate, phosphoromolybdate, phosphorotungstate, phosphorotungstomolybdate, benzenesulphonate, naphtalenesulphonate, 4-chloro-benzenesulphonate, oxalate, maleate, acetate, propionate, lactate, succinate, chloroacetate, tartrate, methanesulphonate or benzoate. Preferable examples of such anions are chloride, hydrogensulphate, sulphate, methosulphate, phosphate, formate, lactate or acetate, especially chloride and methosulphate. The anion can be exchanged in a known manner for another anion.

One class of preferred compounds of formula (1) is that in which the residues A₁ and A₂ are identical, B₁ and B₂ are identical and D₁ and D₂ are identical and, more particularly, compounds of formula (1) in which the moieties
B₁ and/or B₂ are represented by the formulae (2) and/or (3) and in which
R₁ represents hydrogen, a straight-chain C₁-C₄alkyl or branched C₃-C₄alkyl group which may be interrupted by one or two heteroatoms and is unsubstituted or substituted by one or two —OH, —OC₁-C₄alkyl, —NH₂, —NHC₁-C₄alkyl, —N(C₁-C₄alkyl)₂, -N-pyrrolidino, -N-piperidino, -N-morpholino or —N⁺(C₁-C₄alkyl)₃ groups,
A₁ and A₂ are both —SO₃⁻ or —SO₃M,
M, R₂, D₁ and D₂ being as previously defined.

Compounds of formula (1), which are of especial interest, are those in which the moieties
B₁ and B₂ are identical and represented by the formulae (2) or (3), whereby
R₁ represents hydrogen, a straight-chain C₁-C₄alkyl or branched C₃-C₄alkyl group which may be unsubstituted or substituted by one or two —OH, —OC₁-C₄alkyl, —NH₂, —NHC₁-C₄alkyl, —N(C₁-C₄alkyl)₂, -N-pyrrolidino, -N-piperidino, -N-morpholino or —N⁺(C₁-C₄alkyl)₃ group,
R₂ represents C₁-C₄alkyl,
A₁ and A₂ are both —SO₃⁻ or —SO₃M, whereby
M represents hydrogen, potassium or sodium and
D₁ and D₂ are identical and may be represented by halogen, especially chlorine,
C₁-C₄monoalkyl- or dialkylamino, said alkyl groups being unsubstituted or substituted by mono- or di-C₁-C₄alkylamino or tri-C₁-C₄alkylammonium; C₂-C₄hydroxyalkylamino, C₂-C₄-di(hydroxyalkyl)amino, anilino, an aniline sulphonamide residue or a morpholino-, piperidino- or —N—C₁-C₄substituted piperazino ring.

Most especially preferred compounds of formula (1), in which B₁ and B₂ are identical and represented by the formulae (2), are those in which
R₁ represents hydrogen, a straight-chain C₁-C₄alkyl, especially methyl, ethyl or n-propyl, or branched C₃-C₄alkyl group which may be unsubstituted or substituted by one —OH, for example hydroxyethyl or hydroxypropyl, —N(C₁-C₄alkyl)₂, especially dimethyl- or diethylamino, -N-pyrrolidino, or —N⁺(C₁-C₄alkyl)₃ group, or by one —OH group and one —N⁺(C₀-C₄alkyl)₃ group, for example, 3-trimethylammonium-2-hydroxy-1-propyl,
A₁ and A₂ are both —SO₃⁻ or —SO₃M, whereby
M represents hydrogen, potassium or sodium and
D₁ and D₂ are identical and may be represented by chlorine, C₁-C₄monoalkyl-, for example methyl-, ethyl or propylamino, or dialkylamino, for example dimethyl or diethylamino, said alkyl groups being unsubstituted or substituted by di-C₁-C₄alkylamino, for example dimethylaminopropylamino, or tri-C₁-C₄alkylammonium, for example trimethylammoniumpropylamino; C₂-C₄hydroxyalkylamino, especially hydroxyethyl or hydroxypropyl, C₂-C₄-di(hydroxyalkyl)amino, in particular, diethanolamino, anilino, an aniline 4-sulphonamide residue or a morpholino-, or —N—C₁-C₄substituted piperazino, for example an N-methyl piperazino, ring.

Most especially preferred compounds of formula (1), in which B₁ and B₂ are identical and represented by the formulae (3), are those in which
R₁ represents hydrogen, methyl, ethyl or hydroxyethyl,
R₂ represents hydrogen, methyl or ethyl,
A₁ and A₂ are both —SO₃⁻ or —SO₃M, whereby
M represents hydrogen, potassium or sodium and
D₁ and D₂ are identical and may be represented by dimethylaminopropylamino, trimethylammoniumpropylamino; C₂-C₄hydroxyalkylamino, diethanolamino, anilino, an aniline 4-sulphonamide residue or a morpholino-, or —N—C₁-C₄substituted piperazino, for example an N-methyl piperazino or an N,N-dimethylpiperazinium, ring.

A further class of preferred compounds of formula (1) is that in which the residues A₁ and A₂ are identical, B₁ and B₂ are identical and D₁ and D₂ are identical and, more particularly, compounds of formula (1) in which the moieties
B₁ and/or B₂ are represented by the formulae (4) and/or (5), whereby
R₄ represents the group —X′—NR₆R₇ or the group —X′—N⁺R₃R₆R₇,
X and X′ each, independently of each other, represent a straight-chain C₂-C₈alkylene or branched C₃-C₈alkylene chain, which is unsubstituted or substituted by one or two —OH or —C(—O)— groups,
R₃ and R₅ each, independently of each other, represent hydrogen, C₁-C₄alkyl or C₂-C₄hydroxyalkyl,
R₆ and R₇ each, independently of each other, represent hydrogen, C₁-C₄alkyl or, together with the nitrogen atom to which they are bound, complete a pyrrolidino, piperidino or morpholino ring,
A₁ and A₂ are both —SO₃⁻ or —SO₃M,
M, R₂, D₁ and D₂ being as previously defined.

More especially preferred are compounds of formula (1) in which the moieties
B₁ and B₂ are identical and represented by the formulae (4) or (5) are those in which R₄ represents the group —X′—NR₆R₇ or the group —X′—N⁺R₃R₆R₇,
X and X′ each, independently of each other, represent a C₂-C₄alkylene chain, which is unsubstituted or substituted by —OH,
R₃ and R₅ each, independently of each other, represent hydrogen or C₁-C₄alkyl,
R₆ and R₇ each, independently of each other, represent hydrogen, C₁-C₄alkyl or, together with the nitrogen atom to which they are bound, complete a pyrrolidino, piperidino or morpholino ring,
R₂ represents C₁-C₄alkyl,
A₁ and A₂ are both —SO₃⁻ or —SO₃M, whereby
M represents hydrogen, potassium or sodium and
D₁ and D₂ are identical and may be represented by halogen, especially chlorine, C₁-C₄monoalkyl- or dialkylamino, said alkyl groups being unsubstituted or substituted by mono- or di-C₁-C₄alkylamino or tri-C₁-C₄alkylammonium; C₂-C₄hydroxyalkylamino, C₂-C₄-di(hydroxyalkyl)amino, anilino, an aniline sulphonamide residue or a morpholino-, piperidino- or —N—C₁-C₄alkylsubstituted piperazino ring, an anilino residue being preferred.

Most especially preferred are compounds of formula (1) in which the moieties
B₁ and B₂ are identical and represented by the formulae (4) or (5) are those in which
R₄ represents the group —X′—NR₆R₇ or the group —X′—N⁺R₃R₆R₇,
X and X′ each, independently of each other, represent a propylene chain, which is unsubstituted or substituted by —OH,
R₃ and R₅ each, independently of each other represent hydrogen or methyl,
R₆ and R₇ each represent methyl,
R₂ represents methyl,
A₁ and A₂ are both —SO₃⁻ or —SO₃M, whereby
M represents hydrogen, potassium or sodium and
D₁ and D₂ are identical and represent either an anilino or aniline-4-sulphonamido residue.

Alternatively, one further preferred aspect of the invention relates to a three-component mixture of compounds of formula (1), comprising two symmetrical components, i.e. compounds of formula (1) in which the residues A₁ and A₂ are identical, B₁ and B₂ are identical and D₁ and D₂ are identical, and a third component in which the residues A₁ and A₂ are identical, but either, B₁ and B₂ are different or D₁ and D₂ are different, which, preferably, may be illustrated by the following formulae (1a), (1b) and (1c): 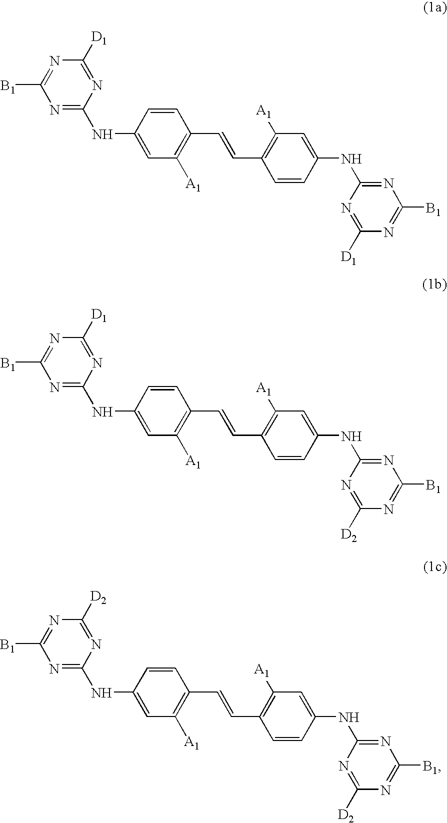
wherein A₁, B₁, D₁ and D₂ and also the preferences thereof are as previously described. Most preferred mixtures, however, are those in which A₁ represents —SO₃H, B₁ represents a 4-(2-hydroxyethyl)piperazin-1-yl residue, D₁ is anilino and D₂ is either a morpholino or a 2-hydroxypropylamino residue.

Within the scope of the definitions of the substituents R₁, R₂, R₃, R₄, R₅, R₆ and/or R₇, straight-chain C₁-C₁₂alkyl groups are, for example, methyl, ethyl, n-propyl, n-butyl, n-pentyl, n-hexyl, n-heptyl, n-octyl, n-nonyl, n-decyl, n-undecyl, and n-dodecyl, whilst branched C₃-C₁₂alkyl groups are, for example, isopropyl, sec-butyl, isobutyl, t-butyl, 2-ethylbutyl, isopentyl, 1-methylpentyl, 1,3-dimethylbutyl, 1-methylhexyl, isoheptyl, 1,1,3,3-tetramethylbutyl, 1-methytheptyl, 3-methylheptyl, 2-ethylhexyl, 1,1,3-trimethylhexyl, 1,1,3,3-tetramethylpentyl, 1-methylundecyl and 1,1,3,3,5,5-hexamethylhexyl. Where the C₂-C₁₂alkyl group is interrupted by heteroatoms, these may be sulphur, nitrogen or, especially, oxygen, whilst C₂-C₄hydroxyalkyl may be hydroxyethyl, hydroxy-n- or isopropyl or hydroxybutyl.

A C₂-C₈alkylene chain, in the definitions of X and X′, may, for example be an ethylene, n-propylene, methyl ethylene, 1- or 2-methylpropylene, n-butylene, ethylethylene, n-pentylene, ethyl propylene, dimethyl propylene, methyl butylene, n-hexylene, dimethyl butylene, methyl pentylene, ethyl butylene, n-heptylene, methyl hexylene, dimethyl pentylene, ethyl pentylene, trimethyl butylene, n-octylene, methyl heptylene, dimethyl or ethyl hexylene or a trimethyl heptylene chain.

Within the scope of the definitions of D₁ and D₂ in formula (1), halogen is iodine, bromine, fluorine or, especially, chlorine, whilst C₁-C₄monoalkyl- or dialkylamino may be, for example, mono- or dimethylamino, mono- or diethylamino, mono- or dipropyl- or butylamino. Tri-C₁-C₄alkylammonium is, for example, trimethylammonium, ethyl dimethylammonium, triethylammonium, methyl diethylammonium, tripropyl or tributylammonium, whilst C₂-C₄hydroxyalkylamino and C₂-C₄di(hydroxyalkyl)amino may be, for example, ethanolamino, diethanolamino, propanolamino, dipropanolamino, hydroxybutylamino or di(hydroxybutyl)amino and a 5- or 6-membered, saturated heterocyclic ring is, for example, pyrrolidino, morpholino, piperidino or piperazino.

Where M represents an alkaline or alkaline earth metal, this may be lithium, potassium, sodium, calcium or magnesium, whilst alkylammonium may be ammonium which is mono-, di-, tri- or tetrasubstituted by C₁-C₄alkyl or C₂-C₄hydroxyalkyl or a mixture thereof.

The compounds of formula (1) of the invention may be prepared by reacting, under known reaction conditions, cyanuric chloride, successively, in any desired sequence, with each of 4,4′-diaminostilbene-2,2′-disulphonic acid, an amino compound capable of introducing groups B₁ and/or B₂ or precursors thereof and an amino compound capable of introducing groups D₁ and/or D₂ or precursors thereof, B₁, B₂, D₁ and D₂ being as previously defined. The mixtures of compounds of the invention may be obtained purely by mechanical mixing of the individual components or, preferably, from the synthesis by employing mixtures of amino compounds capable of introducing groups B₁ and/or B₂ or precursors thereof and or mixtures of amino compounds capable of introducing groups D₁ and/or D₂ or precursors thereof.

In certain cases it may be advantageous to firstly produce an intermediate compound by way of the above reaction sequence, which is subsequently further reacted to result in the compound of formula (1). Thus, for example, a compound of formula (1) in which B₁ and/or B₂ is represented by formula (3) may be obtained by firstly preparing the corresponding compound in which B₁ and/or B₂ is represented by formula (2) and subsequent reaction with a compound capable of introducing the group R₂. Similarly, a compound of formula (1) in which B₁ and/or B₂ is represented by formula (5) may be obtained from the corresponding compound in which B₁ and/or B₂ is represented by formula (4). Furthermore, such compounds may also be obtained from the corresponding amine precursors by treatment with suitable reactants capable of introducing any one of the groups R₁-R₅. Suitable reactants are, for example, alkylating or quaternising agents such as dimethyl or diethyl sulphate, chloro or bromoacetic acids, esters or amides, appropriate alkyl chlorides, bromides or iodides or compounds capable of introducing the group —X′—NR₆R₇ or —X′—NR₃R₆R₇ such as 3-chloro-2-hydroxy-1,1,1-trimethyl propylammonium chloride or with analogous compounds. In this respect, certain compounds designated above as intermediates are novel and, as a consequence, a further aspect of the invention are compounds of the formula 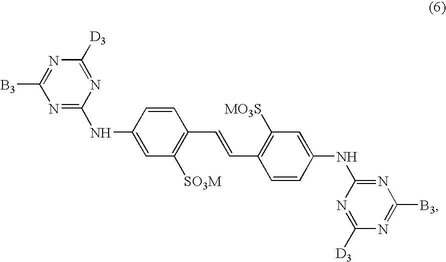
wherein
B₃ represents a group of the formula —NH(CH₂)ₙNR₈R₉, n being 2, 3 or 4 and
D₃ represents halogen, an anilino, anilino-sulphonic acid or anilino-sulphonamide residue, R₈ and R₉ each. Independently of each other, represent hydrogen, C₁-C₄alkyl, C₂-C₄-hydroxyalkyl or, together with the nitrogen atom to which they are bound, complete a pyrrolidino, piperidino or morpholino ring and M, is as defined in claim 1, with the proviso that those compounds in which D₃ is anilino, B₃ is an N-(3-aminopropyl)-diethanolamino, N,N-dimethyl-1,3-propanediamino or 4-(3′-aminopropyl)morpholine residue or in which D₃ represents a sulphanilamide residue and B₃ is a 4-(3′-aminopropyl)morpholine residue and M is hydrogen are excluded, which are useful as intermediates for the preparation of compounds of formula (1).

Compounds of formula (6), which are of special interest, are those in which
B₃ represents a group of the formula —NH(CH₂)₃NHR₈R₉,
D₃ represents an anilino or anilino-4-sulphonamide residue,
M represents hydrogen or sodium and
R₈ and R₉ are hydrogen or C₁-C₄alkyl, preferably hydrogen.

The compounds of formula (6) of the invention may be similarly prepared by reacting, under known reaction conditions, cyanuric chloride, successively, in any desired sequence, with each of 4,4′-diaminostilbene-2,2′-disulphonic acid, an amino compound capable of introducing groups B₃ and an amino compound capable of introducing groups D₃, B₃ and D₃ being as previously defined.

All starting materials are known compounds, which are readily available or may be prepared by known methods.

A further aspect of the invention is a composition for whitening synthetic or natural organic materials, which contains water, a fluorescent whitening agent of formula (1) or mixtures thereof and, optionally, auxiliaries.

More specifically, such brightener compositions contain water and, in each case based on the weight of the formulation, from 3 to 25% by weight, preferably from 5 to 15% by weight of the above defined fluorescent whitening agent mixture and also 0 to 60%, preferably 5 to 50% by weight, of auxiliaries.

Suitable auxiliaries include, for example, anionic or non-ionic dispersants from the class of ethylene oxide adducts with fatty alcohols, higher fatty acids or alkyl phenols or ethylenediamine ethylene oxide-propylene oxide adducts, copolymers of N-vinylpyrrolidone with 3-vinylpropionic acid, water retention aids, such as ethylene glycol, glycerol or sorbitol, or biocides.

Most of the compounds of formula (1) and their mixtures are excellent fluorescent whitening agents for substrates such as textiles, for the addition to detergent compositions and, especially for the fluorescent whitening of paper.

Accordingly, the present invention further provides a method for the fluorescent whitening of a substrate comprising contacting the substrate with a compound having the formula (1) or mixtures of compounds.

When used for the fluorescent whitening of paper, the compound of formula (1) and the mixtures according to the present invention may be applied to the paper substrate in the pulp mass, in the form of a paper coating composition, or directly in the size press or metering press.

In one preferred aspect, the present invention provides a method for the fluorescent whitening of a paper surface, comprising contacting the paper surface with a coating composition comprising a white pigment; a binder dispersion; optionally a water-soluble co-binder; and sufficient of a fluorescent whitening agent having the formula (1) or mixtures thereof according to the present invention, to ensure that the treated paper contains 0.01 to 1% by weight, based on the white pigment, of a fluorescent whitening agent having the formula (1).

As the white pigment component of the paper coating composition used according to the method of the present invention, there are preferred inorganic pigments, e.g., aluminium or magnesium silicates, such as China clay and kaolin and, further, barium sulfate, satin white, titanium dioxide, calcium carbonate (chalk) or talcum; as well as white organic pigments.

The paper coating compositions used according to the method of the present invention may contain, as binder, inter alia, plastics dispersions based on copolymers of butadiene/styrene, acrylonitrile/butadiene/styrene, acrylic acid esters, acrylic acid esters/styrene/acrylonitrile, ethylene/vinyl chloride and ethylene/vinyl acetate; or homopolymers, such as polyvinyl chloride, polyvinylidene chloride, polyethylene and polyvinyl acetate or polyurethanes. A preferred binder consists of styrene/butyl acrylate or styrene/butadiene/acrylic acid copolymers or styrene/butadiene rubbers. Other polymer lattices are described, for example, in U.S. Pat. Nos. 3,265,654, 3,657,174, 3,547,899 and 3,240,740.

The optional water-soluble protective colloid may be, e.g., soya protein, casein, carboxymethylcellulose, natural or modified starch, chitosan or a derivative thereof or, especially, polyvinyl alcohol. The preferred polyvinyl alcohol protective colloid component may have a wide range of saponification levels and molecular weights; e.g. a saponification level ranging from 40 to 100; and an average molecular weight ranging from 10,000 to 100,000.

Recipes for coating compositions for paper are described, for example, in J. P. Casey “Pulp and Paper”; Chemistry and Chemical Technology, 2nd edition, Volume III, pages 1684-1649 and in “Pulp and Paper Manufacture”, 2nd and 5th edition, Volume II, page 497 (McGraw-Hill).

The paper coating compositions used according to the method of the present invention preferably contain 10 to 70% by weight of a white pigment. The binder is preferably used in an amount, which is sufficient to make the dry content of polymeric compound up to 1 to 30%, by weight, preferably 5 to 25% by weight, of the white pigment. The amount of fluorescent brightener preparation used according to the invention is calculated so that the fluorescent brightener is preferably present in amounts of 0.01 to 1% by weight, more preferably 0.05 to 1% by weight, and especially 0.05 to 0.6% by weight, based on the white pigment.

The paper coating composition used in the method according to the invention can be prepared by mixing the components in any desired sequence at temperature from 10 to 100° C., preferably 20 to 80° C. The components here also include the customary auxiliaries, which can be added to regulate the rheological properties, such as viscosity or water retention capacity, of the coating compositions. Such auxiliaries are, for example, natural binders, such as starch, casein, protein or gelatin, cellulose ethers, such as carboxyalkylcellulose or hydroxyalkylcellulose, alginic acid, alginates, polyethylene oxide or polyethylene oxide alkyl ethers, copolymers of ethylene oxide and propylene oxide, polyvinyl alcohol, water-soluble condensation products of formaldehyde with urea or melamine, polyphosphates or polyacrylic acid salts.

The coating composition used according to the method of the present invention is preferably used to produce coated printed or writing paper, or special papers such as ink-jet or photographic papers, or cardboard.

The coating composition used according to the method of the invention can be applied to the substrate by any conventional process, for example with an air blade, a coating blade, a roller, a doctor blade or a rod, or in the size press, after which the coatings are dried at paper surface temperatures in the range from 70 to 200° C., preferably 90 to 130° C., to a residual moisture content of 3-8%, for example with infra-red driers and/or hot-air driers. Comparably high degrees of whiteness are thus achieved even at low drying temperatures.

By the use of the method according to the invention, the coatings obtained are distinguished by optimum distribution of the dispersion fluorescent brightener over the entire surface and by an increase in the level of whiteness thereby achieved, by a high fastness to light and to elevated temperature (e.g. stability for 24 hours at 60-100° C.) and excellent bleed-fastness to water.

In a second preferred aspect, the present invention provides a method for the fluorescent whitening of a paper surface comprising contacting the paper in the size press with an aqueous solution containing a size, optionally an inorganic or organic pigment and 0.1 to 20 g/l of a fluorescent whitening agent having the formula (1) or mixtures thereof. Preferably, the size is starch, a starch derivative or a synthetic sizing agent, especially a water-soluble copolymer.

The compounds and mixtures of compounds of the present invention are particularly advantageous in that they exhibit not only extremely high whitening ability, also in the presence of cationic polymers or residual amounts of anionic FWA's, but, in addition, in many cases highly desirable water solubilities and fastness properties.

The following Examples serve to illustrate the invention without intending to be restrictive in nature; parts and percentages are by weight, unless otherwise stated.

### EXAMPLE 1

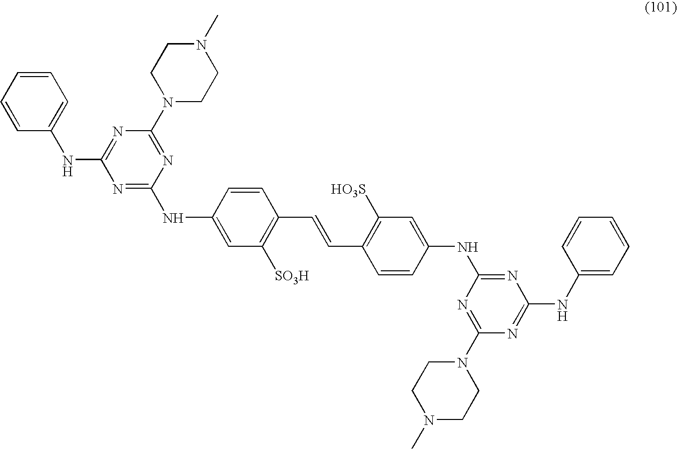

To 24.0 g of 1-methylpiperazine previously heated to 90° C. are added 8.2 g of 4,4′-bis[(4-anilino-6-chloro-1,3,5-triazin-2-yl)amino]stilbene-2,2′-disulphonic acid disodium salt with stirring. The temperature rises to 115° C. and a cloudy solution results. After stirring for 90 minutes at 115-120° C. and then cooling, 25 ml of water are added and the resulting solution evaporated to dryness under vacuum. The product is suspended in 150 ml of water, the pH adjusted to 1 and the mixture stirred for 2 hours. After standing overnight, the product is filtered, washed with 5% sodium chloride solution and dried under vacuum at 70° C. There are obtained 9.0 g of the compound of formula (101) as yellow crystals.

### EXAMPLE 2

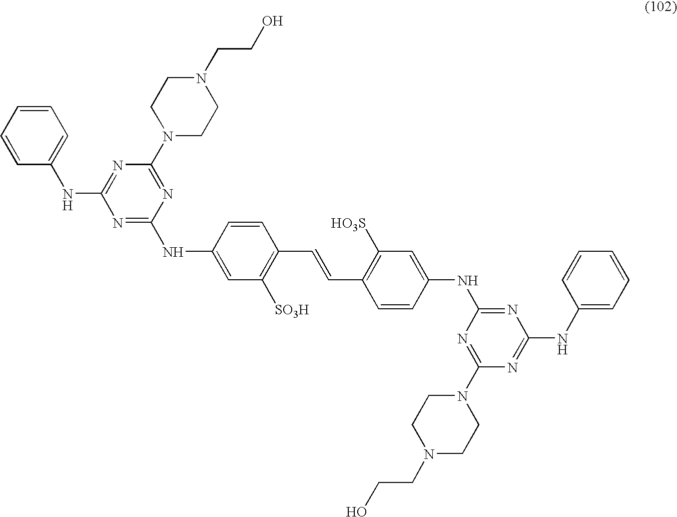

By proceeding in a manner analogous to that described in Example 1, but replacing the 1-methylpiperazine by an equivalent quantity of N-(2-hydroxyethyl)piperazine, the compound of formula (102) is obtained.

### EXAMPLE 3

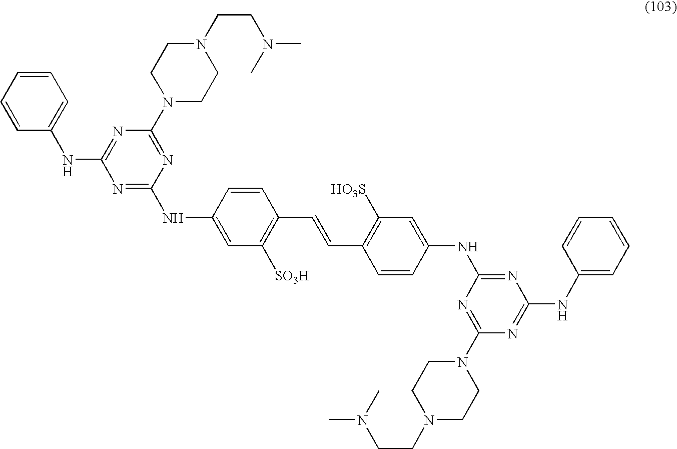

By proceeding in a manner analogous to that described in Example 1, but replacing the 1-methylpiperazine by an equivalent quantity of 1-(2-dimethylaminoethyl)piperazine, the compound of formula (103) is obtained.

### EXAMPLE 4

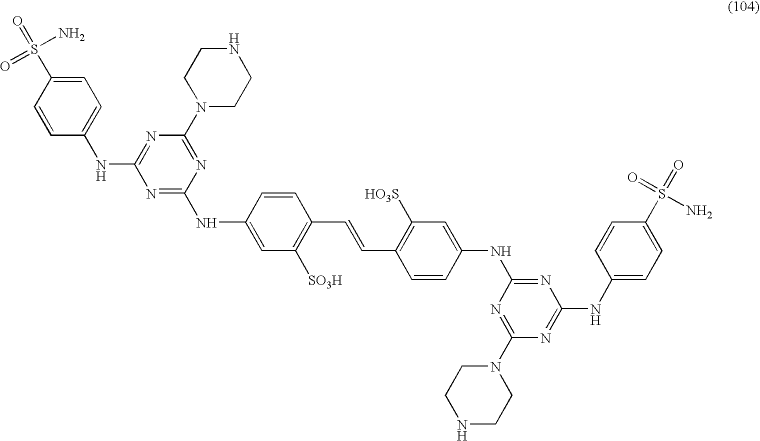

30 g of piperazine are dissolved in 200 ml of water at 8000 under an atmosphere of nitrogen. To the resulting stirred solution, there are added 39.0 g of 4,4′-bis[(4p-sulphonamidoanilino-6-chloro-1,3,5-triazin-2-yl)amino]stilbene-2,2′-disulphonic acid disodium salt at 85-90° C. over 1 hour. After stirring for a further 90 minutes, the solution is cooled to 70° C. and the pH adjusted to 5 by addition of 40 ml of concentrated hydrochloric acid. The precipitate is filtered, washed with water and resuspended in 300 ml of water. After warming to 80° C., 7 ml of 50% sodium hydroxide solution are added whereby the pH rises to 10 and a solution results. The solution is cooled, the pH adjusted to 5 by addition of 18 ml of 17% aqueous hydrochloric acid and the yellow crystalline precipitate filtered off, washed with water and dried under vacuum at 70° C. There are obtained 34.4 g of the compound of formula (104) as yellow crystals.

### EXAMPLE 5

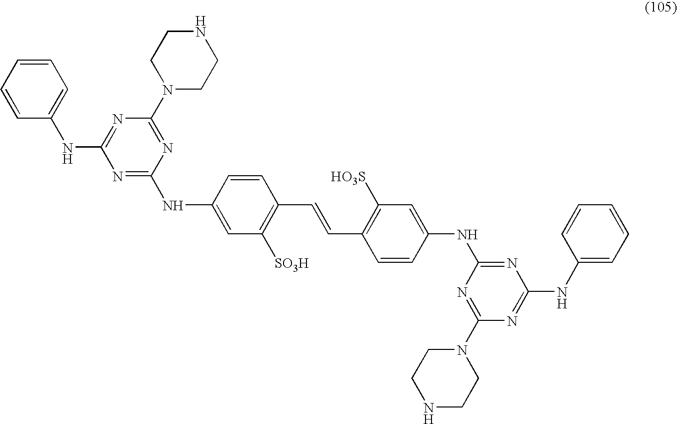

By proceeding in a manner analogous to that described in Example 4, but replacing the 4,4′-bis[(4-p-sulphonamidoanilino-6-chloro-1,3,5-triazin-2-yl)amino]stilbene-2,2′-disulphonic acid disodium salt by an equivalent quantity of 4,4′-bis[(4-anilino-6-chloro-1,3,5-triazin-2-yl)amino]stilbene-2,2′-disulphonic acid disodium salt, the compound of formula (105) is obtained.

### EXAMPLE 6

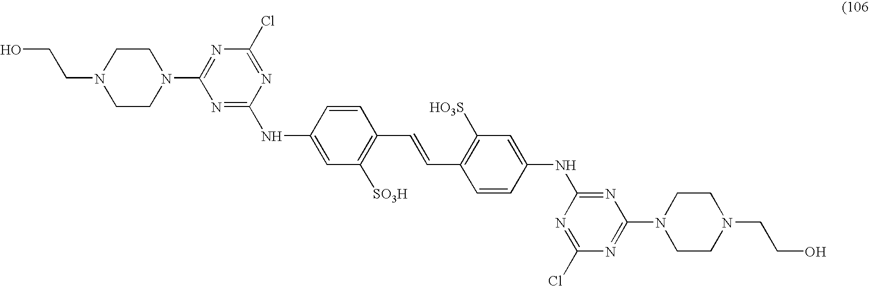

A solution of 120 g of cyanuric chloride in 930 ml of methyl ethyl ketone is added with stirring to 400 g of ice water, with cooling, at 5-10° C. 996 ml of an aqueous solution containing 12 g of 4,4′-diaminostilbene-2,2′-disulphonic acid and 2.5 g of sodium carbonate per 100 ml are then added dropwise during 70 minutes, with stirring, at 5-10° C., the pH being maintained at 4.5-5.5 by addition of aqueous sodium carbonate solution containing 20 g of sodium carbonate per 100 ml. Following the addition, the mixture is stirred for a further 10 minutes at 5-10° C. and then treated dropwise with 86.3 g of N-(2-hydroxyethyl)piperazine during 10 minutes when the pH rises to 8.7 and the temperature to 18° C. The resulting viscous yellow suspension is then warmed to 72° C. over 1 hour and stirring continued at this temperature for a further 2 hours. The temperature is then raised to 85° C. and the methyl ethyl ketone distilled off. The mixture is then cooled to 50° C., allowed to stand overnight, then filtered and washed with 500 ml of water, then with 500 ml of 5% aqueous sodium chloride. After drying at 70° C. under vacuum, there are obtained 295.7 g of the compound of formula (106) as yellow crystals.

### EXAMPLE 7

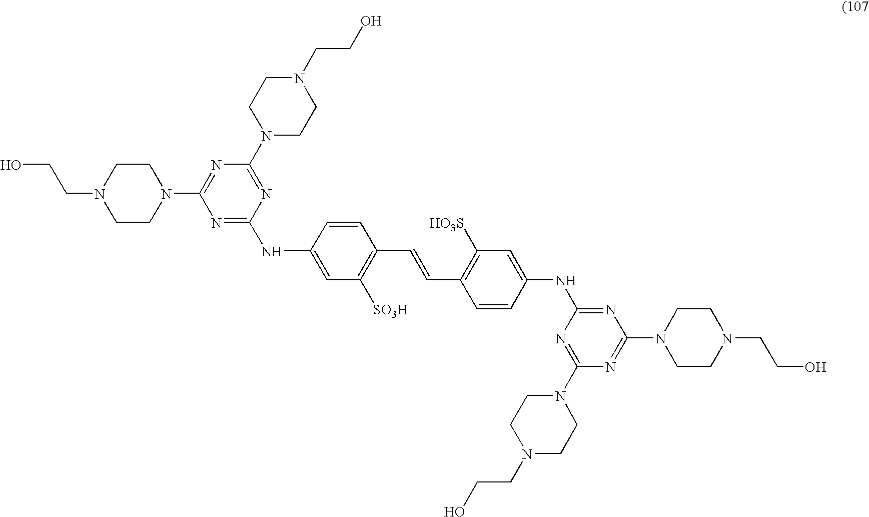

The procedure of Example 5 is repeated, but prior to distillation of the methyl ethyl ketone, 102 g of N-(2-hydroxyethyl)piperazine are added dropwise to the suspension over 15 minutes. The reaction mixture is warmed to 85° C. The pH is adjusted to 8.0-8.5 by addition of an aqueous sodium hydroxide solution containing 50 g of sodium hydroxide per 100 ml and the methyl ethyl ketone distilled off over 90 minutes. During this time the temperature is raised to 97° C. and the mixture stirred for a further 90 minutes at this temperature, the pH being maintained between 8.0 and 8.5 by further addition of aqueous sodium hydroxide. The reaction mixture is cooled to 60° C., filtered with suction and washed with 5% aqueous sodium chloride solution. After drying at 70° C. under vacuum, there are obtained 369 g of the compound of formula (107) as a yellowish white solid.

### EXAMPLE 8

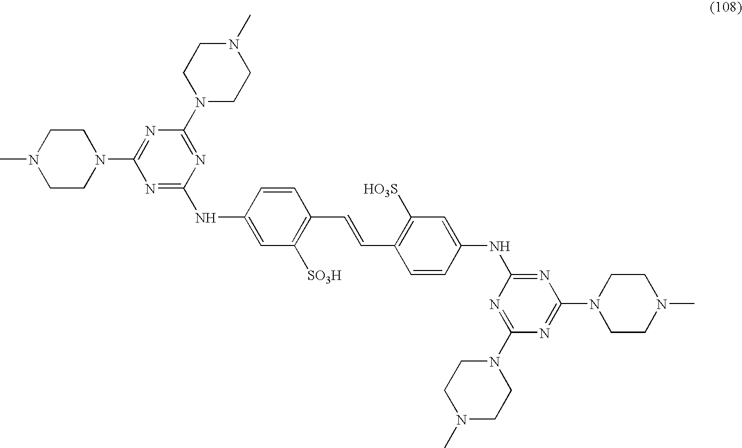

By following the procedure described in Example 7, but replacing the N-(2-hydroxyethyl)piperazine by an equivalent quantity of N-methylpiperazine, there are obtained 255.3 g of the compound of formula (108) as yellow crystals.

### EXAMPLE 9

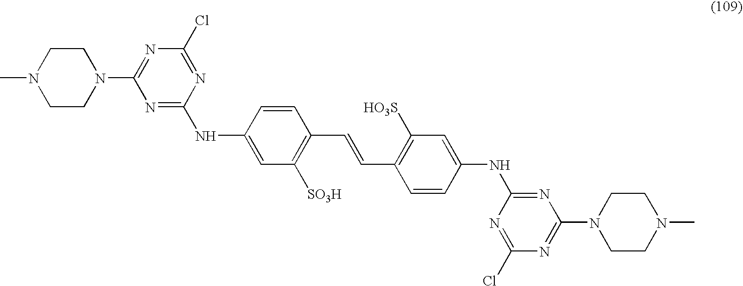

By following the procedure described in Example 6, but replacing the N-(2-hydroxyethyl)piperazine by an equivalent quantity of N-methylpiperazine, there are obtained 246.3 g of the compound of formula (109) as yellow crystals with an active content of 92.9%.

### EXAMPLE 10

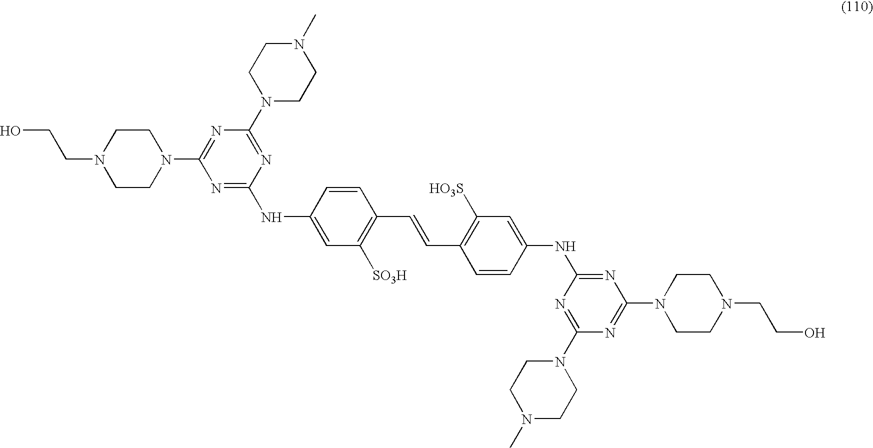

To 45.1 g of N-methylpiperazine previously heated to 80° C., 24.2 g of the compound of formula (106) are added with, stirring over 15 minutes. The temperature is then raised to 115-120° C. and the yellowish brown solution stirred for a further 4 hours at this temperature. After cooling the mixture is diluted with 100 ml of water and the resulting solution evaporated to dryness on a rotary evaporator. This procedure is repeated twice, the residue dissolved in 250 ml of water, the pH adjusted to 1 by addition of concentrated hydrochloric acid, filtered at pH 4.5 and the filter residue washed with 5% sodium chloride solution. After drying at 70° C. under vacuum, there are obtained 27.6 g of the compound of formula (110) as yellowish brown crystals.

### EXAMPLE 11

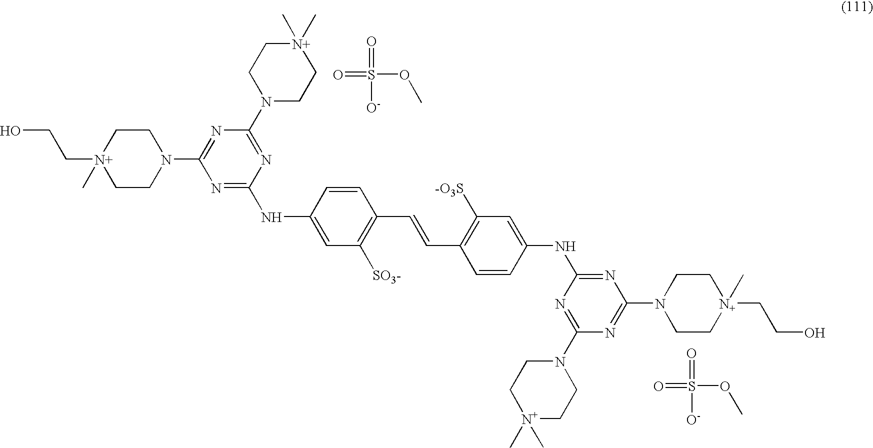

To a mixture of 120 ml of water and 22.5 ml of 2N aqueous sodium hydroxide solution, 14.0 g of the compound of formula (110) are added with stirring at 40° C. The mixture is warmed to 80° C. and the resulting solution then cooled to 45° C. and treated with 15.1 g of dimethyl sulphate over 1-2 minutes. Following the addition, the solution is stirred for 45 minutes at 48° C., the pH being maintained at 10.5-11.0 by addition of a total of 35 ml of 2N aqueous sodium hydroxide solution. The temperature is then raised to 60° C., the mixture stirred for a further 45 minutes at this temperature and then allowed to stand. The reaction mixture is evaporated on a rotary evaporator and the residue dried under vacuum at 70° C. to yield 17.4 g of the compound of formula (111) as yellow crystals.

### EXAMPLE 12

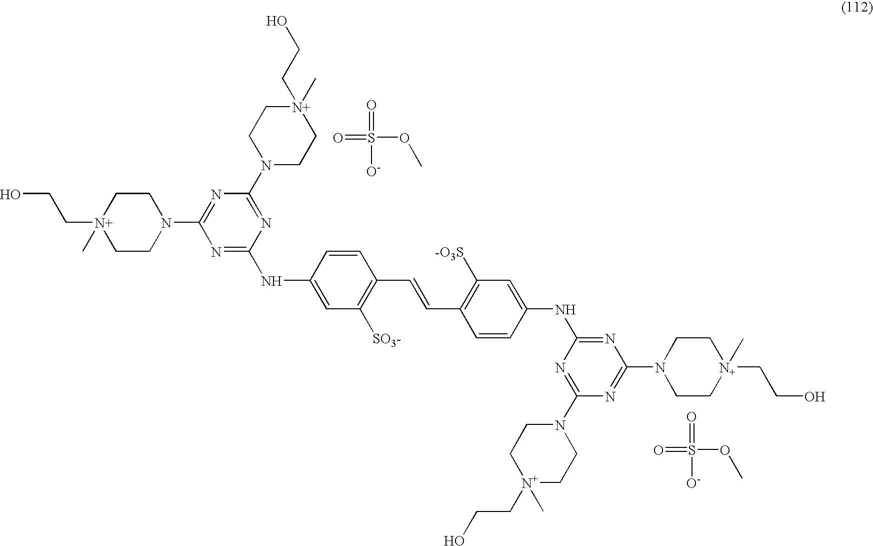

By following the procedure described in Example 11, but replacing the compound of formula (110) by the compound of formula (107), the compound of formula (112) is obtained.

### EXAMPLE 13

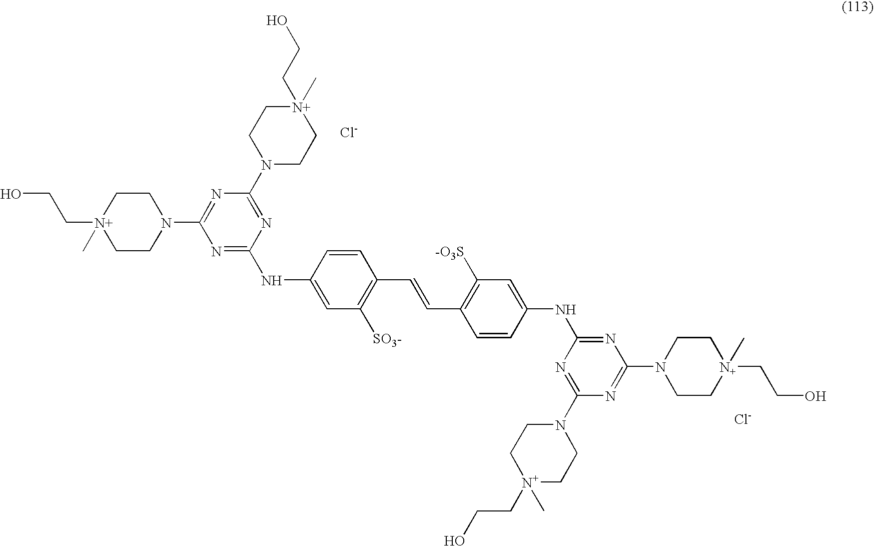

33.0 g of the compound of formula (112) is suspended in 200 ml of an aqueous solution containing 10 g of sodium chloride per 100 ml of solution at room temperature and the pH adjusted to 1 by addition of concentrated hydrochloric acid. The pH is then raised to 5.5 by addition of aqueous sodium hydroxide solution and the resulting suspension stirred for a further 2 hours. The solids are filtered, washed with 10% aqueous sodium chloride solution and dried under vacuum at 70° C. There are obtained 28 g of the compound of formula (113).

### EXAMPLE 14

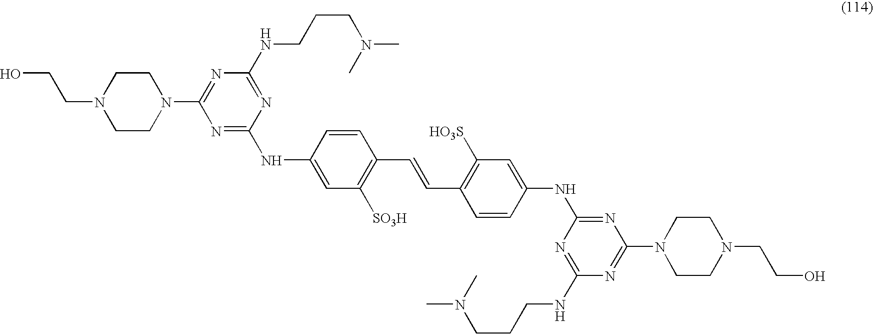

By following the procedure described in Example 10, but replacing the N-methylpiperazine by an equivalent quantity of 3-dimethylamino-n-propylamine, there are obtained 11.4 g of the compound of formula (114) as a yellow powder.

### EXAMPLE 15

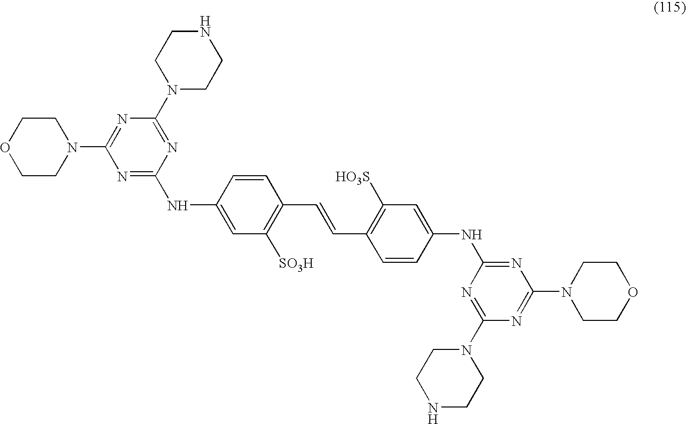

By following the procedure described in Example 5, but replacing the 4,4′-bis[(4-p-sulphonamidoanilino-6-chloro-1,3,5-triazin-2-yl)amino]stilbene-2,2′-disulphonic acid disodium salt by an equivalent quantity of 4,4′-bis[(4-N-morpholino-6-chloro-1,3,5-triazin-2-yl)amino]stilbene-2,2′-disulphonic acid disodium salt, the compound of formula (115) is obtained.

### EXAMPLE 16

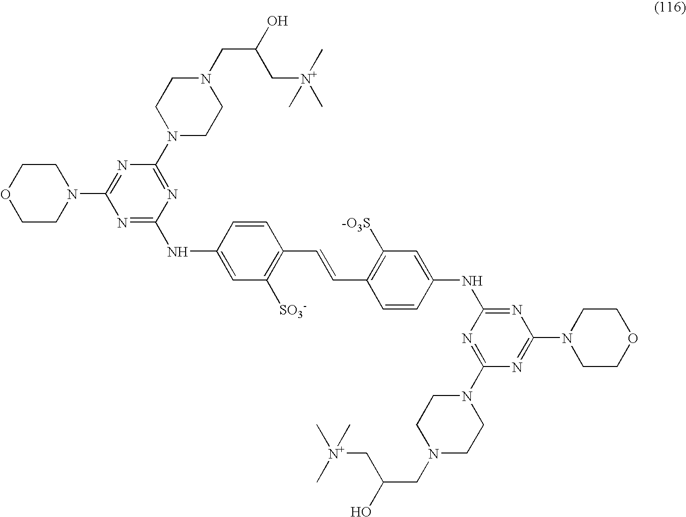

8.67 g of the compound of formula (115) are dissolved in 80 ml of water and 20 ml of 2N aqueous sodium hydroxide solution at 50° C. To this solution are then added 5.78 g of 65% (3-chloro-2-hydroxypropyl)trimethylammonium chloride over 2 minutes and the mixture stirred for 90 minutes at 50° C. A further 0.58 g of 65% (3-chloro-2-hydroxypropyl)-trimethylammonium chloride are then added, stirring continued for 60 minutes, the mixture again treated with 0.58 g of 65% (3-chloro-2-hydroxypropyl)-trimethylammonium chloride, stirred for another 50 minutes at 50° C., the mixture cooled and stirring discontinued. After the solids have settled, the supernatant liquid is decanted and the white residue suspended in 100 ml of water. The pH is first adjusted to 3.5 by addition of concentrated hydrochloric acid and then raised to 9.0 by addition of 2N aqueous sodium hydroxide solution. The precipitate is filtered, the residue slurried 3 times in water and, after the final filtration, dried under vacuum at 70° C. There are obtained 9.6 g of the compound of formula (116) as yellow crystals.

### EXAMPLE 17

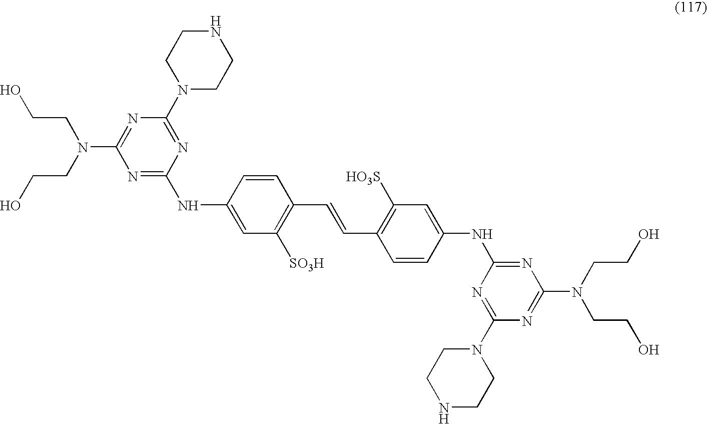

By following the procedure described in Example 5, but replacing the 4,4′-bis[(4-p-sulphonamidoanilino-6-chloro-1,3,5-triazin-2-yl)amino]stilbene-2,2′-disulphonic acid disodium salt by an equivalent quantity of 4,4′-bis[(4-diethanolamino-6-chloro-1,3,5-triazin-2-yl)amino]stilbene-2,2′-disulphonic acid disodium salt, the compound of formula (117) is obtained as a fine yellowish powder.

### EXAMPLE 18

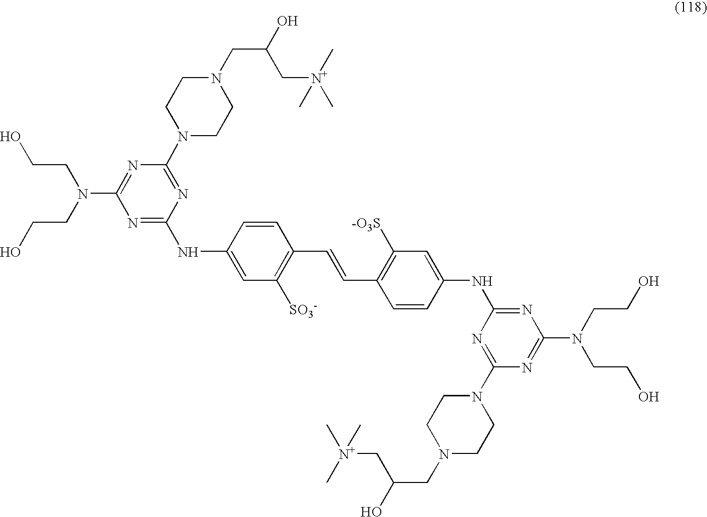

By following the procedure described in Example 16, but replacing the compound of formula (115) by an equivalent quantity of the compound of formula (117), the compound of formula (118) is obtained as a yellowish powder.

### EXAMPLE 19

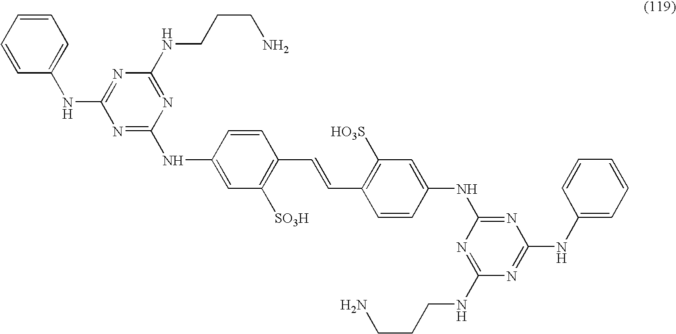

By following the procedure described in Example 1, but replacing the 1-methylpiperazine by an equivalent quantity of 1,3-diamino-n-propane, the compound of formula (119) is obtained as yellow crystals.

### EXAMPLE 20

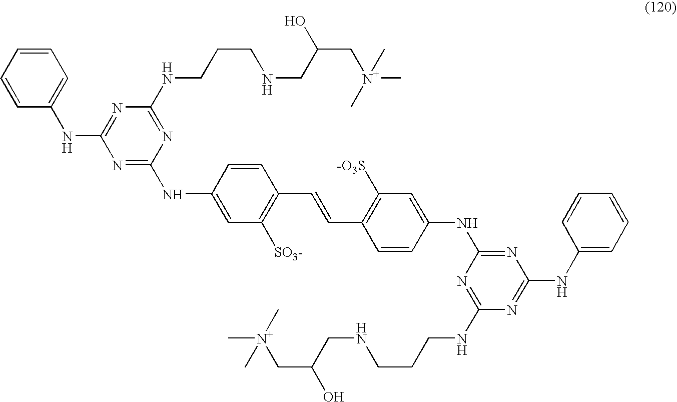

By following the procedure described in Example 16, but replacing the compound of formula (115) by an equivalent quantity the compound of formula (119), the compound of formula (120) is obtained as yellow crystals.

### EXAMPLE 21

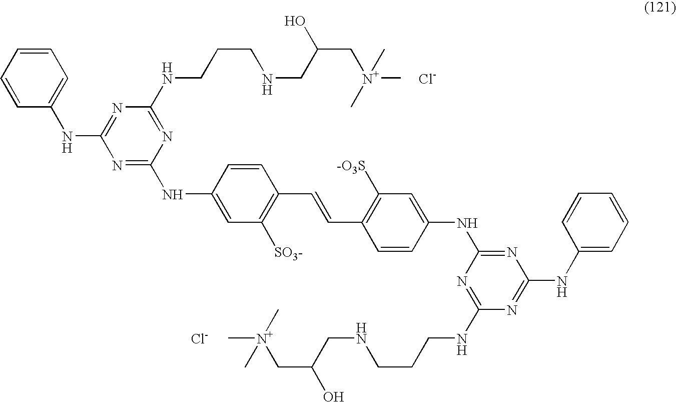

By following the procedure described in Example 16, but replacing the compound of formula (115) by an equivalent quantity the compound of formula (119) and finally adjusting the pH to 4 by addition of concentrated hydrochloric acid, the compound of formula (121) is obtained as yellow crystals.

### EXAMPLE 22

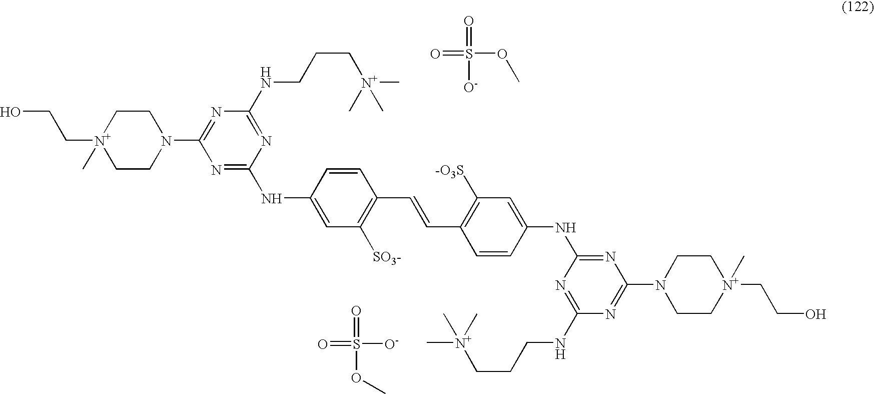

5.0 g of the compound of formula (114) are added to 50 ml of water and 7.5 ml of 2N aqueous sodium hydroxide solution and warmed to 45° C. when a yellow solution results. The solution is cooled to 35° C. and rapidly treated with 3.8 g of dimethyl sulphate. After stirring for 4.5 hours at 35° C., during which time the pH is maintained at 9.0 by addition of a total of 5 ml of 2N aqueous sodium hydroxide solution, the temperature is raised to 60° C. and the mixture stirred for a further 1 hour. The mixture is then evaporated on a rotary evaporator and the residue dried under vacuum at 70° C. There are obtained 8.9 g of the compound of formula (122) as yellow crystals.

### EXAMPLE 23

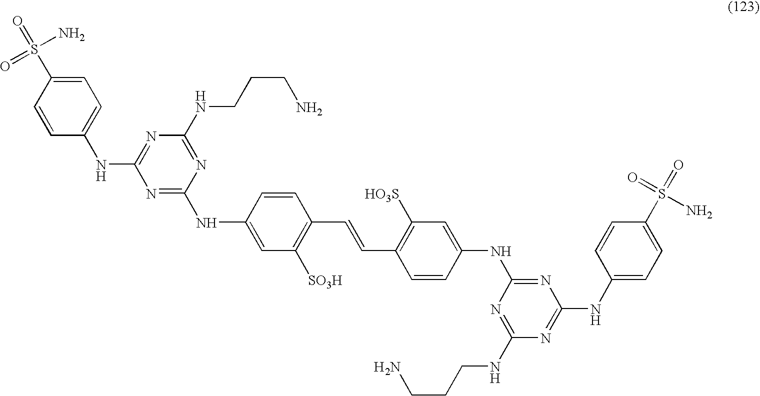

By following the procedure described in Example 5, but replacing the piperazine by an equivalent quantity of 1,3-diaminopropane the compound of formula (123) is obtained as brownish crystals.

### EXAMPLE 24

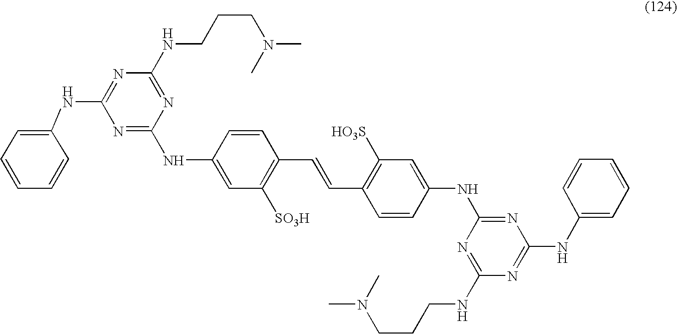

By following the procedure described in Example 1, but replacing the 1-methylpiperazine by an equivalent quantity of 3-N,N′-dimethylamino-1-propylamine, the compound of formula (124) is obtained.

### EXAMPLE 25

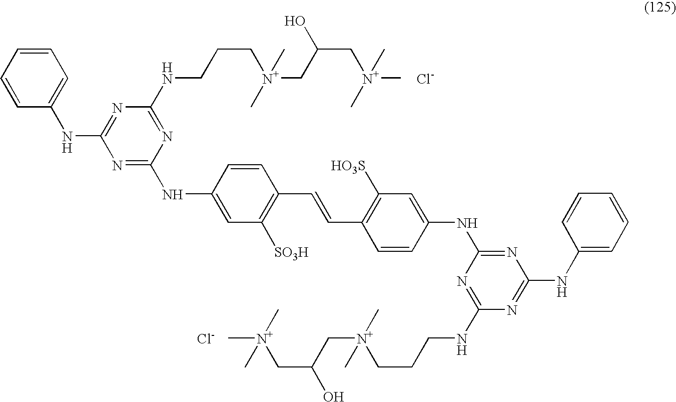

By following the procedure described in Example 16, but replacing the compound of formula (115) by an equivalent quantity of the compound of formula (124), the compound of formula (125) is obtained as a yellowish solid.

### EXAMPLE 26

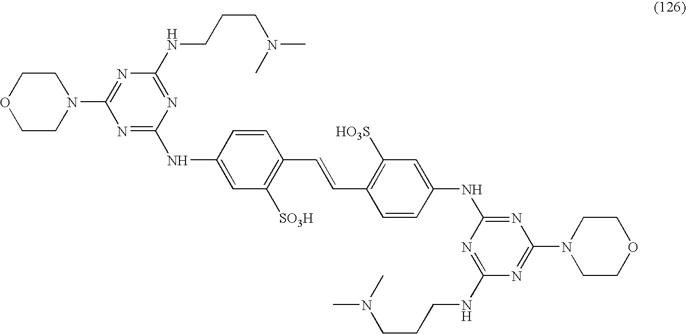

150 g of 4,4′-bis[(4-N-morpholino-6-chloro-1,3,5-triazin-2-yl)amino]stilbene-2,2′-disulphonic acid disodium salt with an active content of 91% are added in portions with stirring at 25° C. to 350 ml of 3-N,N′-dimethylamino-1-propylamine. During the addition, the temperature is raised to 90° C. within 10 minutes and, during the following 10 minutes, further increased to 100° C. and the mixture maintained at this temperature for a further 1 hour. Heating is then ceased and the mixture diluted with 250 ml of water and evaporated under vacuum. The resulting syrup is stirred with 170 ml of concentrated hydrochloric acid for 1 hour at 25° C. and the precipitated solids filtered with suction and dried under vacuum at 70° C. There are obtained 142 g of the compound of formula (126).

### EXAMPLE 27

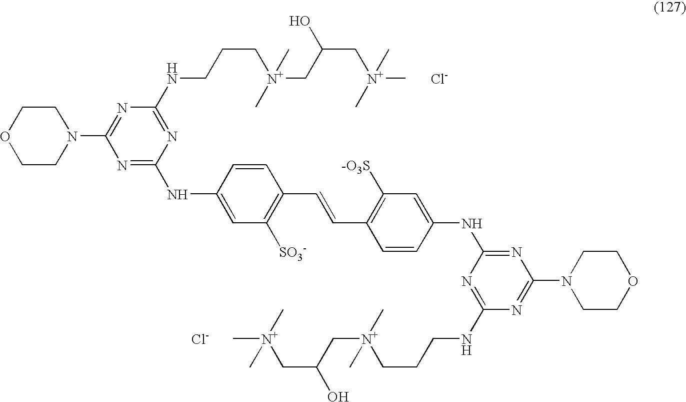

By following the procedure described in Example 16, but replacing the compound of formula (115) by an equivalent quantity of the compound of formula (126), the compound of formula (127) is obtained as a yellow solid.

### EXAMPLE 28

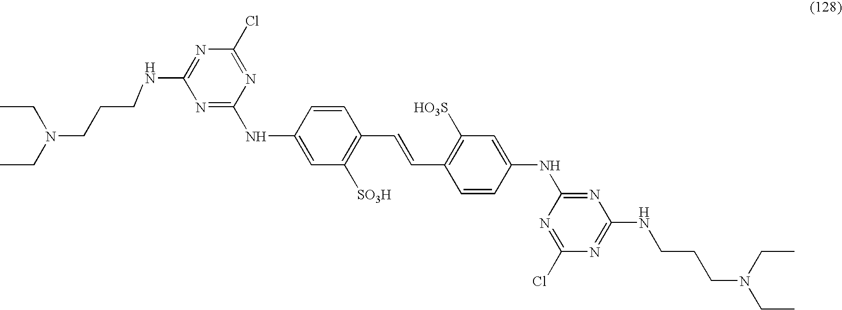

By following the procedure described in Example 6, but replacing the N-(2-hydroxyethyl)piperazine by an equivalent quantity of 3-diethylamino propylamine, 263.7 g of the compound of formula (128) are obtained as yellow crystals.

### EXAMPLE 29

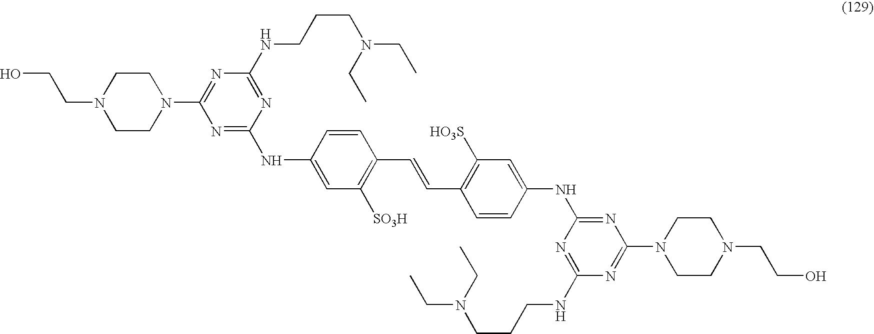

30.0 g of the compound of formula (128) are added to 70 ml of water, the pH adjusted to 10.6 by addition of 4N aqueous sodium hydroxide solution and the mixture heated to 70° C., when solution results. To this solution, 21.9 g of 1-(2-hydroxyethyl)piperazine are added at 70-75° C., the temperature is raised to 96° C. and the solution stirred for a total of 3 hours at this temperature. After cooling to 70° C., the pH is adjusted to 4.5 with concentrated hydrochloric acid, when an oil separates. The residue, after decantation of the aqueous liquors, is treated with 400 ml of water and allowed to stand. 10 g of sodium chloride are added and the solids separated by filtration, washed with 10% brine and dried under vacuum at 70° C. There are obtained 15.8 g of the compound of formula (129) as yellow crystals.

### EXAMPLE 30

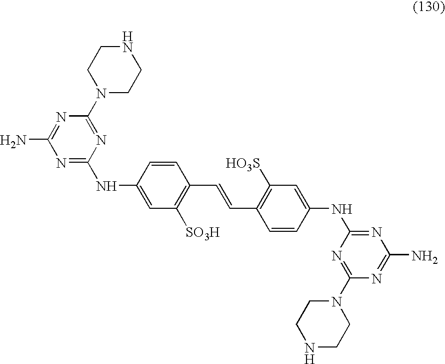

To 58.4 g of piperazine, 150 ml of dioxan and 150 ml of water, previously heated to 70-75° C., are added 40 g of 4,4′-bis[(4-amino-6-chloro-1,3,5-triazin-2-yl)amino]stilbene-2,2′-disulphonic acid disodium salt with stirring. The temperature is raised to 80-85° C. and the yellowish brown solution stirred for 2 hours. The pH is adjusted to 4.5 by addition of 100 ml of concentrated hydrochloric acid, the mixture cooled to 20° C. and the precipitated solids filtered. After suspension in water, dissolution at pH 11 and reprecipitation with concentrated hydrochloric acid at pH 4.5, the filtered solids are washed with water and dried under vacuum at 70° C. There are obtained 20.0 g of the compound of formula (130) as yellow crystals.

### EXAMPLE 31

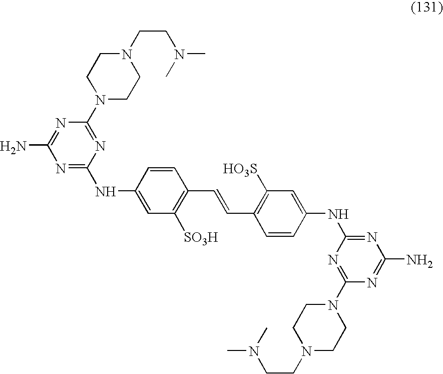

By proceeding essentially as described in Example 30, but replacing the piperazine by an equivalent quantity of 1-(2-dimethylaminoethyl)piperazine, the compound of formula (131) is obtained as yellow crystals.

### EXAMPLE 32

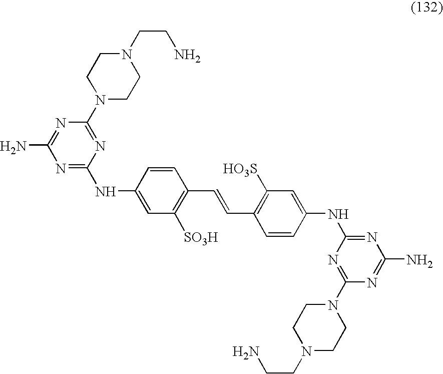

By proceeding essentially as described in Example 30, but replacing the piperazine by an equivalent quantity of 1-(2-aminoethyl)piperazine, the compound of formula (132) is obtained as yellow crystals.

### EXAMPLE 33

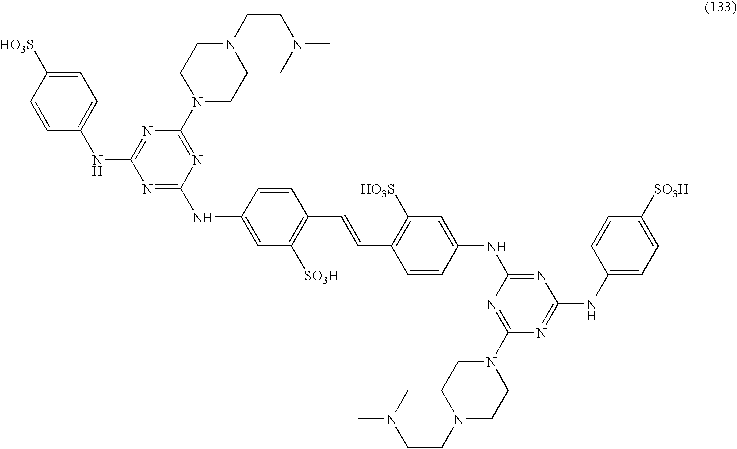

25.0 g of 4,4′-bis[(4-p-sulphoanilino-6-chloro-1,3,5-triazin-2-yl)amino]stilbene-2,2′-disulphonic acid tetra sodium salt are dissolved in 150 ml of water and 8.2 g of 1-(2-dimethylaminoethyl)piperazine added. The temperature is raised to 96° C. and the yellow solution stirred for 3 hours at this temperature, the pH being maintained at 8.3 by addition of 2N aqueous sodium hydroxide solution. After cooling to 70° C., the pH is adjusted to 4.5 by addition of 8 ml of concentrated hydrochloric acid and the mixture stirred for 1 hour. The precipitated solids are filtered, suspended in 250 ml of water and dissolved by addition of sodium hydroxide solution to pH 8.5. Stirring is continued for 2 hours, after which time the pH is adjusted to 4.0 with concentrated hydrochloric acid, the solids filtered, washed with water and dried under vacuum at 70° C. There are obtained 25.5 g of the compound of formula (133) as yellow crystals.

### EXAMPLE 34

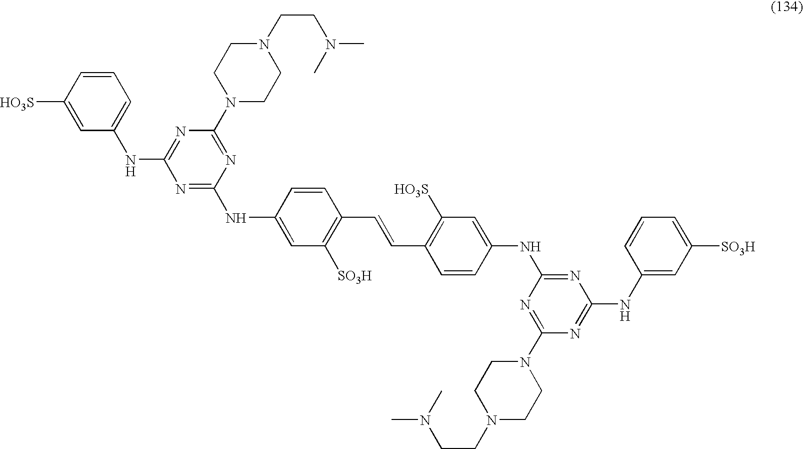

By following the procedure described in Example 33, but replacing the 4,4′-bis[(4-p-sulphoanilino-6-chloro-1,3,5-triazin-2-yl)amino]stilbene-2,2′-disulphonic acid tetra sodium salt by 4,4′-bis[(4-m-sulphoanilino-6-chloro-1,3,5-triazin-2-yl)amino]stilbene-2,2′-disulphonic acid tetra sodium salt, 23.9 g of the compound of formula (134) are obtained as yellow crystals.

### EXAMPLE 35

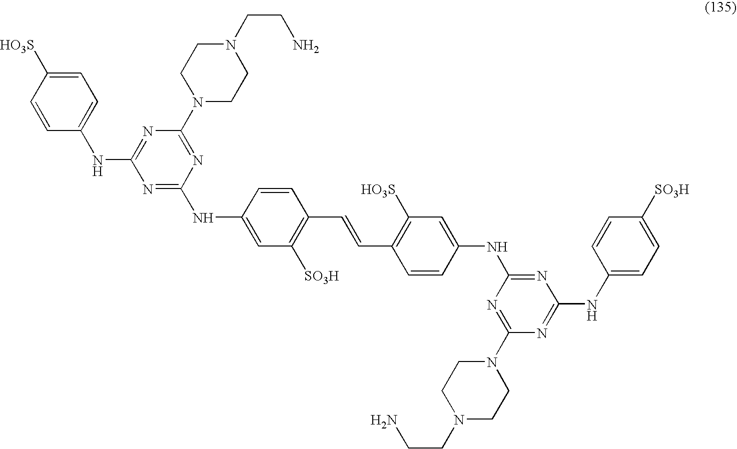

By following the procedure described in Example 33, but replacing the 1-(2-dimethylaminoethyl)-piperazine by an equivalent quantity of 1-(2-aminoethyl)piperazin, 23.8 g of the compound of formula (135) are obtained as yellow crystals.

### EXAMPLE 36

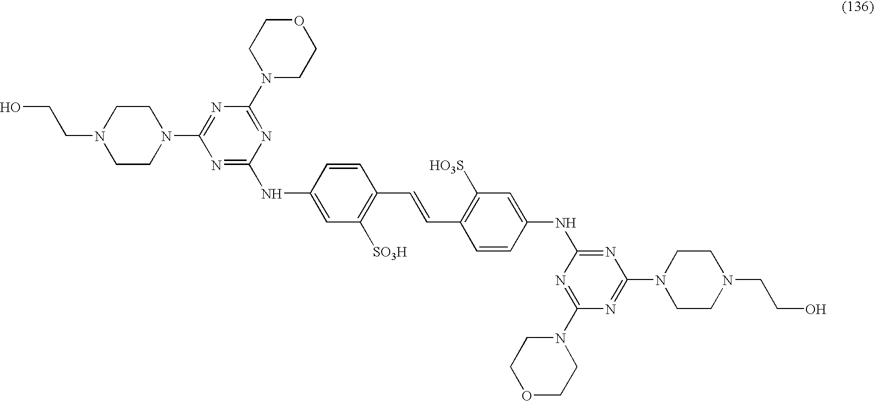

By following the procedure described in Example 7, but replacing the N-(2-hydroxyethyl)piperazine by an equivalent quantity of morpholine, there are obtained 304.9 g of the compound of formula (136) as yellow crystals.

### EXAMPLE 37

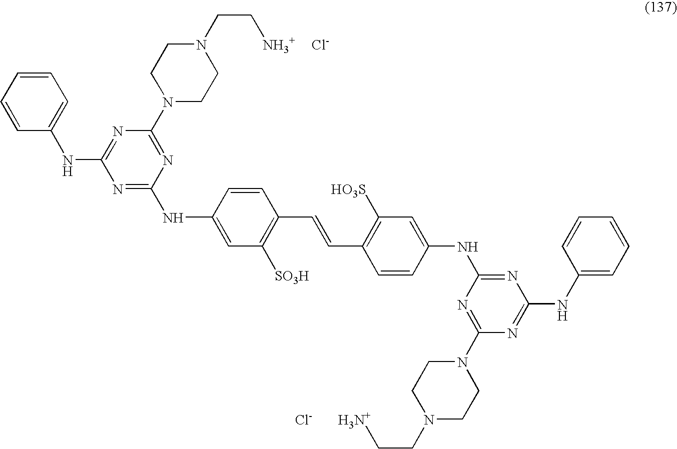

By following the procedure described in Example 30, but replacing the piperazine by an equivalent quantity of 1-(2-aminoethyl)piperazine and the 4,4′-bis[(4-amino-6-chloro-1,3,5-triazin-2-yl)amino]stilbene-2,2′-disulphonic acid disodium salt by an equivalent quantity of 4,4′-bis[(4-anilino-6-chloro-1,3,5-triazin-2-yl)amino]stilbene-2,2′-disulphonic acid disodium salt, 40.3 g of the compound of formula (137) are obtained as yellow crystals.

### EXAMPLE 38

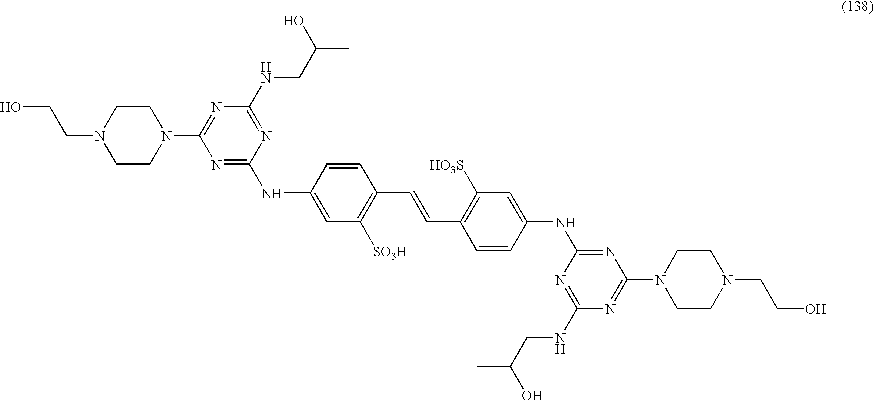

By following the procedure described in Example 30, but replacing the piperazine by an equivalent quantity of 1-(2-hydroxyethyl)piperazine and the 4,4′-bis[(4-amino-6-chloro-1,3,5-triazin-2-yl)amino]stilbene-2,2′-disulphonic acid disodium salt by an equivalent quantity of 4,4′-bis{[4-(2-hydroxy)propylamino-6-chloro-1,3,5-triazin-2-yl]amino}stilbene-2,2′-disulphonic acid disodium salt (see Example 41), 36.8 g of the compound of formula (138) are obtained as yellow crystals.

### EXAMPLE 39

A mixture of compounds of formulae

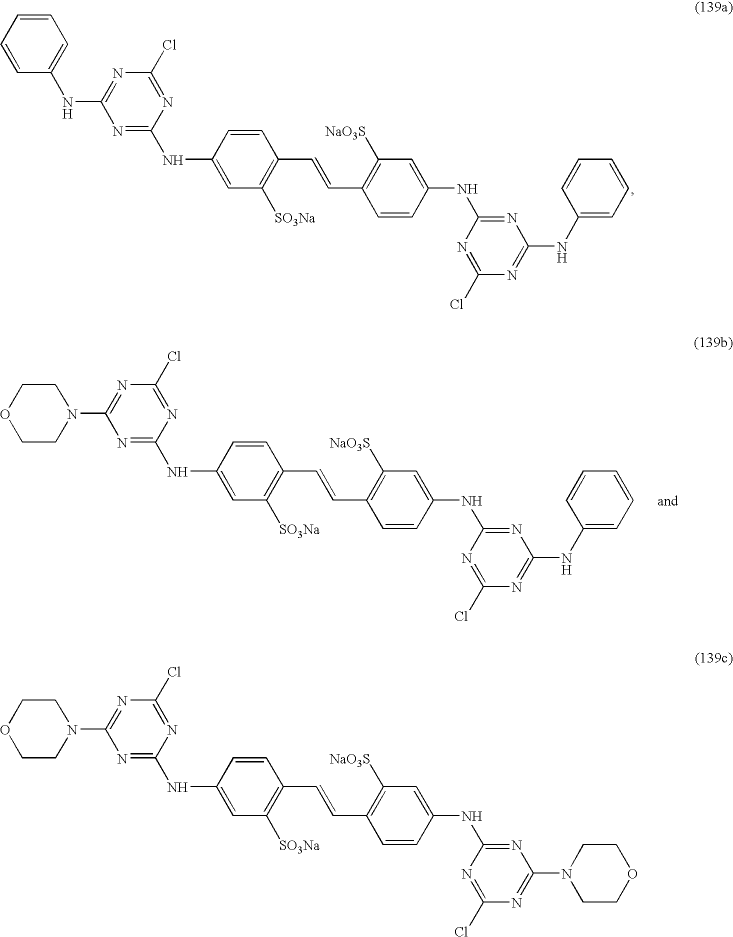

A solution of 120 g of cyanuric chloride in 930 ml of methyl ethyl ketone is added with stirring over 10 minutes at 5-10° C. to 400 g of ice/water. Then, during 70 minutes at a pH of from 4.5 to 5.0, 1093 g of a 12% solution of 4,4′-diaminostilbene-2,2′-disulphonic acid and sodium carbonate are added such that no excess of 4,4′-diaminostilbene-2,2′-disulphonic acid is present. The mixture is stirred for a further 10 minutes at 5-10° C., after which time a total of 21.2 ml of 20% aqueous sodium carbonate solution is consumed. The mixture is warmed to 8-20° C. and the pH adjusted to 7.5 by addition of 50% aqueous sodium hydroxide solution. A mixture of 29.9 g of aniline and 28.0 g of morpholine is then added drop wise over 10 minutes, the mixture warmed to 70° C. during 60 minutes and stirring continued for 90 minutes at this temperature, the methyl ethyl ketone being distilled off. A total of 54.2 ml of 50% aqueous sodium hydroxide solution are required to maintain a pH of 7.5 during this period. The reaction mixture is then cooled to 30° C. over 60 minutes and allowed to stand overnight at room temperature. The supernatant liquid is decanted off, the residue suspended in 750 ml of 5% brine, warmed to 60° C. and then slowly cooled to 30° C. over 60 minutes. The precipitated solids are filtered, washed with 5% brine and dried under vacuum at 70° C. There are obtained 259.1 g of a yellow crystalline product containing 27% of the compound of formula (139a), 46% (139b) and 24% (139c).

### EXAMPLE 40

A mixture of compounds of formulae

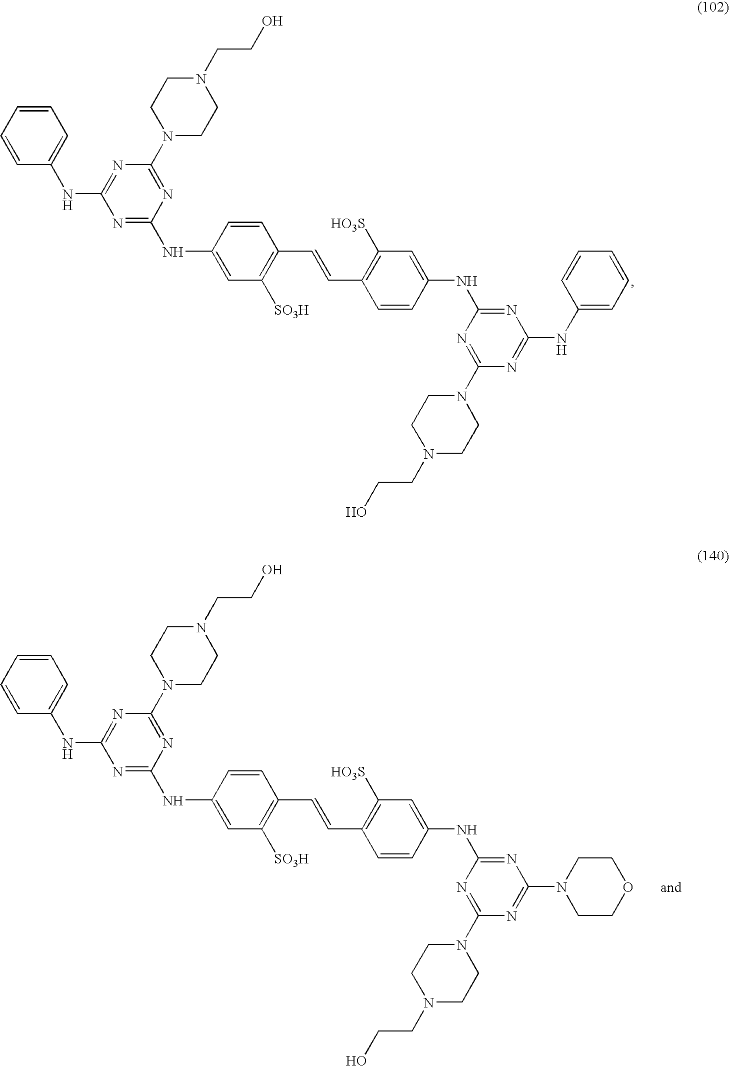

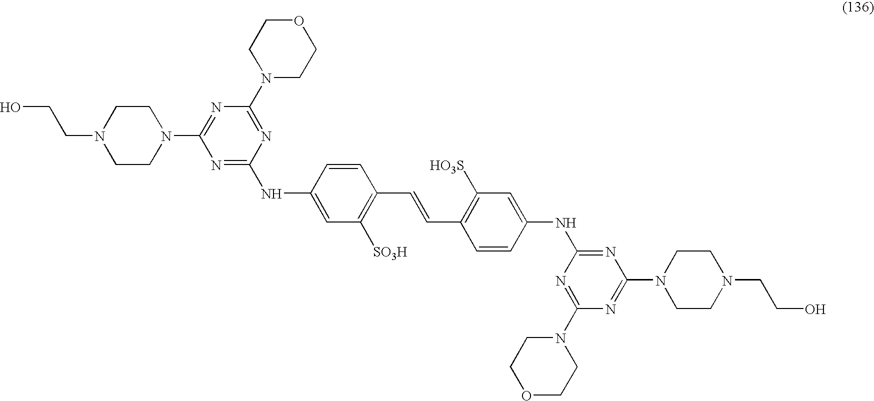

A mixture of 150 ml of water, 150 ml of dioxan and 55.7 g of 1-(2-hydroxyethyl)piperazine is stirred and heated to 70-75° C. and 35.0 g of the mixture of compounds (139a), (139b) and (139c), obtained as described in Example 39, added over 1 hour. The temperature is increased to 86-88° C. and the mixture stirred for a further 4 hours. After cooling to 70° C., 60 ml of concentrated hydrochloric acid are added to pH 4.0 and the mixture further cooled to 20° C. Addition of 100 ml of 5% brine results in precipitation; the solids are filtered, washed with 5% brine and dried under vacuum at 700. There are obtained 26.6 g of a yellow solid consisting of a mixture of 34% of compound (102), 44% of compound (140) and 15% of compound (136).

### EXAMPLE 41

A mixture of compounds of formulae

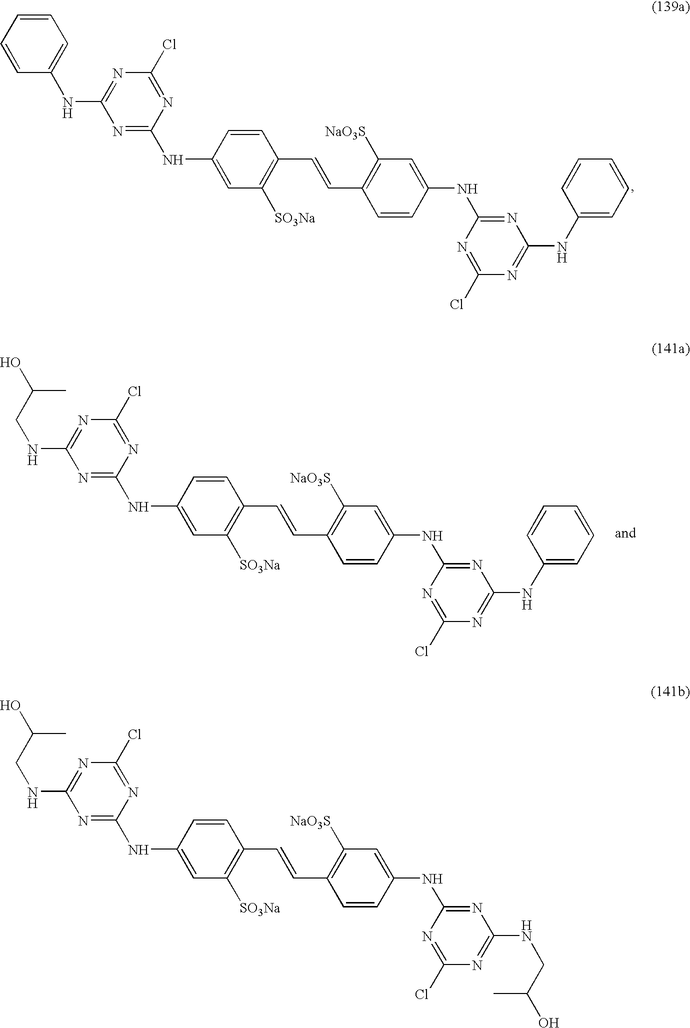

By following the procedure described in Example 139, but replacing the morpholine by an equivalent quantity of 1-aminopropan-2-ol, 188.5 g of a mixture of compounds containing 33% of the compound of formula (139a), 40% (141a) and 23% (141 b) is obtained, as yellow crystals.

### EXAMPLE 42

A mixture of compounds of formulae

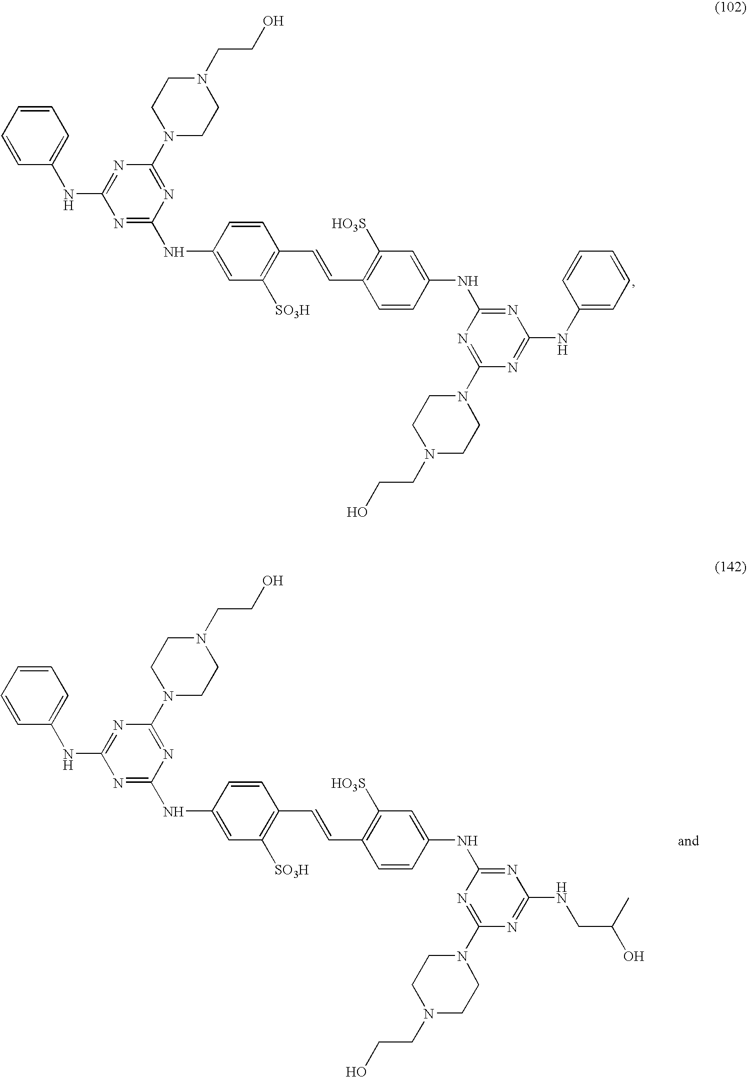

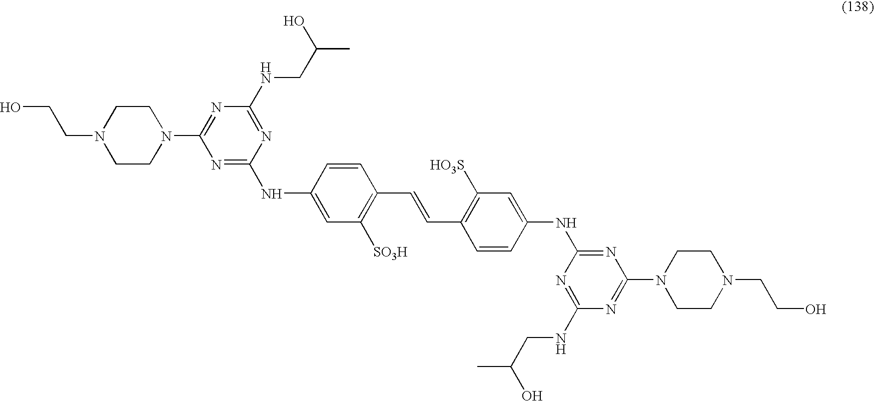

By following the procedure described in Example 40, but replacing the mixture of compounds (139a), (139b) and (139c), obtained as described in Example 39, by an equivalent quantity of the mixture of compounds of formula (139a), (141a) and (141b) of Example 40, there are obtained 35.4 g of yellow crystalline solids containing 35% of the compound of formula (102), 39% (142) and 20% (138).

1. A compound of the formula 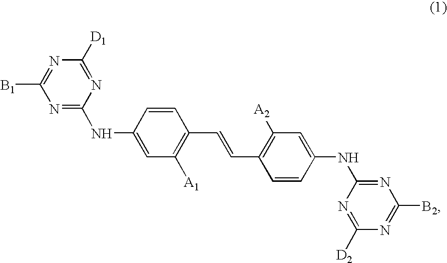
wherein
A₁ and A₂ each, independently of one another, represent —SO₃⁻ or —SO₃M, where
M represents hydrogen, an alkaline or alkaline earth metal, ammonium or alkylammonium,
B₁ and B₂ each, independently of one another, represent the moiety 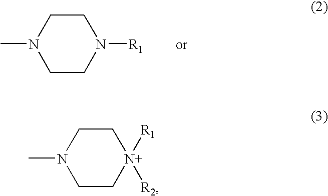
in which
R₁ represents hydrogen, a straight-chain C₁-C₁₂alkyl or branched C₃-C₁₂alkyl group, which C₂-C₁₂alkyl or C₃-C₁₂alkyl group, respectively, may be interrupted by one or two heteroatoms and is unsubstituted or substituted by one or two —OH, —OC₁-C₄alkyl, —NH₂, —NHC₁-C₄alkyl, —N(C₁-C₄alkyl)₂, -N-pyrrolidino, -N-piperidino, -N-morpholino or —N⁺(C₁-C₄alkyl)₃ groups and
R₂ represents C₁-C₄alkyl, C₂-C₄hydroxyalkyl, —CH₂CONH₂, —CH₂COOH or —CH₂COO C₁-C₄alkyl or, alternatively,
B₁ and B₂ each, independently of one another, represent a group of the formula 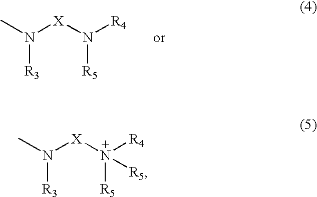
in which
R₃, R₄ and R₅ each, independently of each other, represent hydrogen, C₁-C₄alkyl, C₂-C₄hydroxyalkyl, the group —X′—NR₆R₇ or the group —X′—N⁺R₃R₆R₇, whereby at least one of the substituents R₄ and/or R₅ represents —X′—NR₆R₇ or —X′—N⁺R₃R₆R₇,
X and X′ each, independently of each other, represent a straight-chain C₂-C₈alkylene or branched C₃-C₈alkylene chain, which is unsubstituted or substituted by one or two —OH or —C(═O)— groups,
R₆ and R₇ each, independently of each other, represent hydrogen, C₁-C₄alkyl or, together with the nitrogen atom to which they are bound, complete a pyrrolidino, piperidino or morpholino ring and R₂ is as previously defined and each
D₁ and D₂, independently of one another, are either defined as for B₁ and B₂ or represent halogen, —NH₂, C₁-C₄monoalkyl- or dialkylamino, said alkyl groups being unsubstituted or substituted by C₁-C₄alkoxy, amino, mono- or di-C₁-C₄alkylamino or tri-C₁-C₄alkylammonium;
C₂-C₄hydroxyalkylamino, C₂-C₄di(hydroxyalkyl)amino, anilino, an aniline monosulphonic acid or sulphonamide residue or a 5- or 6-membered, saturated heterocyclic ring or, alternatively, mixtures of compounds of formula (1).

2. A compound of formula (1), according to claim 1, in which the residues A₁ and A₂ are identical, B₁ and B₂ are identical and D₁ and D₂ are identical.

3. A three-component mixture of compounds of formula (1), according to claim 1, comprising two components in which the residues A₁ and A₂ are identical, B₁ and B₂ are identical and D₁ and D₂ are identical, and a third component in which the residues A₁ and A₂ are identical, but either, B₁ and B₂ are different or D₁ and D₂ are different.

4. A compound of formula (1), according to claim 1, in which the moieties B₁ and/or B₂ are represented by the formulae (2) and/or (3) and in which
R₁ represents hydrogen, a straight-chain C₁-C₄alkyl or branched C₃-C₄alkyl group which may be interrupted by one or two heteroatoms and is unsubstituted or substituted by one or two —OH, —OC₁-C₄alkyl, —NH₂, —NHC₁-C₄alkyl, —N(C₁-C₄alkyl)₂, -N-pyrrolidino, -N-piperidino, -N-morpholino or —N⁺(C₁-C₄alkyl)₃ groups,
A₁ and A₂ are both —SO₃⁻ or —SO₃M,
M, R₂, D₁ and D₂ being as defined according to claim 1.

5. A compound of formula (1), according to claim 4, in which the moieties B₁ and B₂ are identical and represented by the formulae (2) or (3), whereby
R₁ represents hydrogen, a straight-chain C₁-C₄alkyl or branched C₃-C₄alkyl group which may be unsubstituted or substituted by one or two —OH, —OC₁-C₄alkyl, —NH₂, —NHC, —C₄alkyl, —N(C₁-C₄alkyl)₂, -N-pyrrolidino, -N-piperidino, -N-morpholino or —N⁺(C₁-C₄alkyl)₃ group,
R₂ represents C₁-C₄alkyl,
A₁ and A₂ are both —SO₃⁻ or —SO₃M, whereby
M represents hydrogen, potassium or sodium and
D₁ and D₂ are identical and may be represented by halogen, especially chlorine, —NH₂, C₁-C₄monoalkyl- or dialkylamino, said alkyl groups being unsubstituted or substituted by mono- or di-C₁-C₄alkylamino or tri-C₁-C₄alkylammonium; C₂-C₄hydroxyalkylamino, C₂-C₄-di(hydroxyalkyl)amino, anilino, an aniline sulphonamide or sulphonic acid residue or a morpholino-, piperidino- or —N—C₁-C₄substituted piperazino ring.

6. A compound of formula (1), according to claim 1, in which the moieties B₁ and/or B₂ are represented by the formulae (4) and/or (5), whereby
R₄ represents the group —X′—NR₆R₇ or the group —X′—N⁺R₃R₆R₇,
X and X′ each, independently of each other, represent a straight-chain C₂-C₈alkylene or branched C₃-C₈alkylene chain, which is unsubstituted or substituted by one or two —OH or —C(═O)— groups, R₃ and R₅ each, independently of each other, represent hydrogen, C₁-C₄alkyl or C₂-C₄hydroxyalkyl, R₆ and R₇ each, independently of each other, represent hydrogen, C₁-C₄alkyl or, together with the nitrogen atom to which they are bound, complete a pyrrolidino, piperidino or morpholino ring,
A₁ and A₂ are both —SO₃⁻ or —SO₃M,
M, R₂, D₁ and D₂ being as defined according to claim 1.

7. A compound of formula (1), according to claim 6, in which the moieties B₁ and B₂ are identical and represented by the formulae (4) or (5) whereby
R₄ represents the group —X′—NR₆R₇ or the group —X′—N⁺R₃R₆R₇,
X and X′ each, independently of each other, represent a C₂-C₄alkylene chain, which is unsubstituted or substituted by —OH,
R₃ and R₅ each, independently of each other, represent hydrogen or C₁-C₄alkyl,
R₆ and R₇ each, independently of each other, represent hydrogen, C₁-C₄alkyl or, together with the nitrogen atom to which they are bound, complete a pyrrolidino, piperidino or morpholino ring,
R₂ represents C₁-C₄alkyl,
A₁ and A₂ are both —SO₃⁻ or —SO₃M, whereby
M represents hydrogen, potassium or sodium and
D₁ and D₂ are identical and may be represented by halogen, C₁-C₄monoalkyl- or dialkylamino, said alkyl groups being unsubstituted or substituted by mono- or di-C₁-C₄alkylamino or tri-C₁-C₄alkylammonium; C₂-C₄hydroxyalkylamino, C₂-C₄-di(hydroxyalkyl)amino, anilino, an aniline sulphonamide residue or a morpholino-, piperidino- or —N—C₁-C₄alkylsubstituted piperazino ring.

8. A process for the preparation of a compound of formula (1) as defined in claim 1, or for mixtures of said compounds comprising two components in which the residues A₁ and A₂ are identical, B₁ and B₂ are identical and D₁ and D₂ are identical, and a third component in which the residues A₁ and A₂ are identical, but either, B₁ and B₂ are different or D₁ and D₂ are different, by reacting, under known reaction conditions, cyanuric chloride, successively, in any desired sequence, with each of 4,4′-diaminostilbene-2,2′-disulphonic acid, an amino compound capable of introducing groups B₁ and/or B₂ or precursors or mixtures thereof and an amino compound capable of introducing groups D₁ and/or D₂ or precursors or mixtures thereof, B₁, B₂, D₁ and D₂ being as defined in claim 1.

9. A compound of the formula 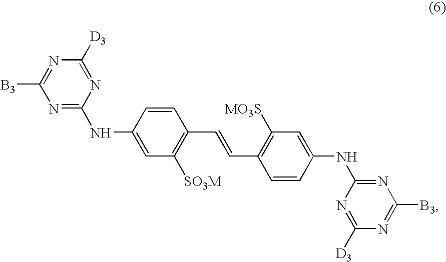
wherein
B₃ represents a group of the formula —NH(CH₂)ₙNR₈R₉, n being 2, 3 or 4 and
D₃ represents halogen, an anilino, anilino-sulphonic acid or anilino-sulphonamide residue,
R₈ and R₉ each independently of each other, represent hydrogen, C₁-C₄alkyl, C₂-C₄-hydroxyalkyl or, together with the nitrogen atom to which they are bound, complete a pyrrolidino, piperidino or morpholino ring and M is as defined in claim 1, with the proviso that those compounds in which D₃ is anilino, B₃ is an N-(3-aminopropyl)-diethanolamino, N,N-dimethyl-1,3-propanediamino or 4-(3′-aminopropyl)morpholine residue or in which D₃ represents a sulphanilamide residue, B₃ is a 4-(3′-aminopropyl)morpholine residue and M is hydrogen are excluded.

10. A process for the preparation of a compound of formula (6) as defined in claim 9 by reacting, under known reaction conditions, cyanuric chloride, successively, in any desired sequence, with each of 4,4′-diaminostilbene-2,2′-disulphonic acid, an amino compound capable of introducing groups B₃ and an amino compound capable of introducing groups D₃, B₃ and D₃ being as defined in claim 9.

11-13. (canceled)

14. A method for optical brightening of synthetic or natural organic materials, which comprises contacting said materials with an aqueous composition comprising a compound of formula (1) as defined in claim 1 or a mixture thereof.

15. A method for optical brightening of paper in pulp, size-press, metering press or coating applications, which comprises contacting said materials with an aqueous composition comprising a compound of formula (1) as defined in claim 1 or a mixture thereof.

16. A method for optical brightening of paper in pulp, size-press, metering press or coating applications, which comprises contacting said materials with an aqueous composition comprising a mixture of compounds of formula (1) as defined in claim 3.
